SEQUENCE LISTING (10 sequences)

SEQ ID NO: 1 - protein, 121 aa, synthetic construct [sequence omitted]

SEQ ID NO: 2 - protein, 5 aa, synthetic construct
  REGION 1..5: note = Description of Artificial Sequence: Synthetic peptide
SYWMH

SEQ ID NO: 3 - protein, 17 aa, synthetic construct
  REGION 1..17: note = Description of Artificial Sequence: Synthetic peptide
EIDPSESNTNYNQKFKG

SEQ ID NO: 4 - protein, 12 aa, synthetic construct
  REGION 1..12: note = Description of Artificial Sequence: Synthetic peptide
GGYDGWDYAIDY

SEQ ID NO: 5 - protein, 112 aa, synthetic construct [sequence omitted]

SEQ ID NO: 6 - protein, 16 aa, synthetic construct
  REGION 1..16: note = Description of Artificial Sequence: Synthetic peptide
RSSQSLAKSYGNTYLS

SEQ ID NO: 7 - protein, 7 aa, synthetic construct
  REGION 1..7: note = Description of Artificial Sequence: Synthetic peptide
GISNRFS

SEQ ID NO: 8 - protein, 9 aa, synthetic construct
  REGION 1..9: note = Description of Artificial Sequence: Synthetic peptide
LQGTHQPYT

SEQ ID NO: 9 - protein, 451 aa, synthetic construct
  REGION 1..451: note = Description of Artificial Sequence: Synthetic peptide
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, synthetic construct
  REGION 1..219: note = Description of Artificial Sequence: Synthetic peptide
DVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0182581 A1
(43) Pub. Date: Jun. 6, 2024

(54) METHODS FOR THE TREATMENT OF CHRONIC POUCHITIS

(71) Applicant: Millennium Pharmaceuticals, Inc., Cambridge, MA (US)

(72) Inventors: Maria Rosario, Cambridge, MA (US); Michael David Laurence Smyth, Berkshire (GB)

(21) Appl. No.: 18/344,724

(22) Filed: Jun. 29, 2023

Related U.S. Application Data
(63) Continuation-in-part of application No. 17/655,541, filed on Mar. 18, 2022.
Application No. 17/655,541 is a continuation of application No. 16/617,254, filed on Nov. 26, 2019, now abandoned.
(60) Provisional application No. 63/367,305, filed on Jun. 29, 2022.
(60) Provisional application No. 62/511,832, filed on May 26, 2017.

(51) Int. Cl. C07K 16/28; A61K 31/496; A61K 39/00; A61K 39/395; A61P 1/00

(52) U.S. Cl. CPC C07K 16/2839; A61K 31/496; A61K 39/3955; A61P 1/00; A61K 2039/505; A61K 2039/545; C07K 2317/24

(57) ABSTRACT

The invention provides methods for the treatment of chronic pouchitis comprising administering an anti-α4β7 antibody, e.g., vedolizumab, to a human subject in need thereof.

### RELATED APPLICATIONS

This application claims the benefit of U.S. Provisional Application No. 63/367,305, filed on Jun. 29, 2022, and this application is a continuation in part of U.S. patent application Ser. No. 17/655,541, filed on Mar. 18, 2022, which is a continuation of U.S. patent application Ser. No. 16/617,254, filed on Nov. 26, 2019, now abandoned, which is a National Stage Entry of International Application No. PCT/IB2018/053760, filed on May 26, 2018, which claims the benefit of U.S. Provisional Application No. 62/511,832, filed on May 26, 2017. The entire contents of the foregoing applications are incorporated herein by reference.

### SEQUENCE LISTING

The instant application contains a Sequence Listing which has been submitted electronically in XML file format and is hereby incorporated by reference in its entirety. Said XML copy, created on Feb. 8, 2024, is named T103022_1040 US_CP1_0588_6_SL.xml and is 14,723 bytes in size.

### FIELD OF THE INVENTION

The present invention relates to the use of an anti-α4β7 integrin antibody, e.g., vedolizumab, for the treatment or prevention of chronic pouchitis.

### BACKGROUND OF THE INVENTION

Pouchitis is a poorly understood condition, with a reported cumulative frequency of 23% to 46% over 10 to 11 years in patients who underwent an ileoanal pouch procedure (Fazio V W, et al. Ileal pouch-anal anastomoses complications and function in 1005 patients. Ann Surg 1995; 222(2): 120-7; Ferrante M, et al. Outcome after proctocolectomy with ileal pouch-anal anastomosis for ulcerative colitis. Inflamm Bowel Dis 2008; 14(1):20-8). The etiology of pouchitis is likely multi-factorial. Pouchitis may be due in part to exposure of the ileal mucosa of the pouch to noxious components of feces, such as short chain fatty acids and bile acids, to which it may never be completely adapted. Altered immunoregulation, previously undiagnosed Crohn's disease (CD) affecting the ileum, and ischemia caused by decreased mucosal blood flow may also be involved in the etiology of pouchitis (Shen B. Acute and chronic pouchitis—pathogenesis, diagnosis and treatment. Nat Rev Gastroenterol Hepatol 2012; 9(6):323-33).

The clinical response of pouchitis to treatment with antibiotics such as metronidazole or ciprofloxacin, as well as probiotics including Lactobacilli, suggests that fecal stasis, C. difficile infection, bacterial overgrowth, or dysbiosis (altered proportions among fecal bacterial populations) may be triggers. Other therapeutic agents tested for the treatment of pouchitis with mixed success include mesalamine, corticosteroids, nutritional agents (short-chain fatty acids, glutamine, or soluble fiber administered per suppository or enema), immunomodifying agents, cigarettes and transdermal nicotine, bisthmus-containing agents, and allopurinol. (Shen B. Acute and chronic pouchitis-pathogenesis, diagnosis and treatment. Nat Rev Gastroenterol Hepatol 2012; 9(6):323-33). Importantly, none of these therapies have been shown to be effective in clinical trials. Furthermore, due to their nonspecific mechanism of action, use of some of these therapies may place patients at risk for infection complications.

Results from 3 clinical trials of the probiotic agent VSL #3 for the primary and secondary prophylaxis of pouchitis have been published and showed efficacy around 85% to 90%. However, long-term efficacy in routine care could not be reproduced in another study.

While pouchitis may respond to short-term antibiotic therapy, some patients experience recurrent pouchitis and require chronic antibiotic therapy to sustain remission or the more drastic option of surgical removal of the pouch. Chronic or recurrent pouchitis is often managed with long-term antibiotic administration, with metronidazole and ciprofloxacin being the antibiotics most often prescribed (Mahadevan et al. Diagnosis and management of pouchitis. Gastroenterology 2003; 124(6):1636-50).

There are presently no approved drugs for the treatment or prevention of pouchitis in the United States or Europe. Thus, there is a large unmet medical need for effective therapies for pouchitis, particularly chronic pouchitis.

### SUMMARY OF THE INVENTION

The invention provided herein discloses, inter alia, methods of treating pouchitis by administering to a subject an anti-α4β7 integrin antibody, e.g., vedolizumab. The invention further provides methods of treating chronic pouchitis by administering to a subject an anti-α4β7 integrin antibody, e.g., vedolizumab.

In one aspect, the invention provides a method of treating chronic pouchitis in a human subject, said method comprising selecting a human subject having chronic pouchitis and administering a therapeutically effective dose of an anti-α4β7 antibody, or antigen binding fragment thereof, to the subject, such that chronic pouchitis is treated, wherein the anti-α4β7 antibody, or antigen binding fragment thereof, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

In another aspect, the invention provides a method of treating chronic pouchitis in a human subject, said method comprising selecting a human subject having chronic pouchitis and administering a therapeutically effective dose of an anti-α4β7 antibody, or antigen binding fragment thereof, to the subject, such that chronic pouchitis is treated, wherein the anti-α4β7 antibody, or antigen binding fragment thereof, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6, and wherein the human subject had an endoscopic Pouchitis Disease Activity Index (PDAI) subscore of 6 at selection and/or was TNFα naïve at selection

In one embodiment, the therapeutically effective dose is selected from the group consisting of 108 mg, 300 mg, and 600 mg.

In another aspect, the invention provides a method of treating chronic pouchitis in a human subject, said method comprising selecting a human subject who has chronic pouchitis; and administering to the human subject an initial dose of 300 mg of an anti-α4β7 antibody, or antigen binding fragment thereof, followed by one or more subsequent doses of 300 mg of the anti-α4β7 antibody, or antigen binding fragment thereof, at least every two weeks, such that the chronic pouchitis is treated in the subject, wherein the anti-α4β7 antibody, or antigen binding fragment thereof, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

In another aspect, the invention includes a method of treating chronic pouchitis in a human subject, said method comprising selecting a human subject who has chronic pouchitis;
administering an initial dose of 300 mg of an anti-α4β7 antibody, or an antigen binding fragment thereof, to the human subject; administering a second dose of 300 mg of the anti-α4β7 antibody, or antigen binding fragment thereof, at about two weeks after the initial dose; administering a third dose of 300 mg of the anti-α4β7 antibody, or antigen binding fragment thereof, at about six weeks after the initial dose; and administering one or more subsequent doses of 300 mg of the anti-α4β7 antibody, or antigen binding fragment thereof, every eight weeks after the third dose, wherein the anti-α4β7 antibody, or antigen binding fragment thereof, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

In yet another aspect, the invention provides a method of treating chronic pouchitis in a human subject, said method comprising selecting a human subject who has chronic pouchitis;
administering an initial dose of 300 mg of an anti-α4β7 antibody, or an antigen binding fragment thereof, to the human subject: administering a second dose of 300 mg of the anti-α4β7 antibody, or antigen binding fragment thereof, at about two weeks after the initial dose; administering a third dose of 300 mg of the anti-α4β7 antibody, or antigen binding fragment thereof, at about six weeks after the initial dose; and administering a dose of 300 mg of the anti-α4β7 antibody, or
antigen binding fragment thereof, every four or eight weeks after the third dose, wherein the anti-α4β7 antibody, or antigen binding fragment thereof, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

In a further aspect, the invention provides a method of treating chronic pouchitis in a human subject, said method comprising selecting a human subject who has chronic pouchitis;
administering an initial dose of 600 mg of an anti-α4β7 antibody, or an antigen binding fragment thereof, to the human subject; administering a second dose of 600 mg of the anti-α4β7 antibody, or antigen binding fragment thereof, at about two weeks after the initial dose; administering a third dose of 600 mg of the anti-α4β7 antibody, or antigen binding fragment thereof, at about six weeks after the initial dose; and administering a dose of 600 mg of the anti-α4β7 antibody, or antigen binding fragment thereof, every four or eight weeks after the third dose, wherein the anti-α4β7 antibody, or antigen binding fragment thereof, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a

CDR1 domain as set forth in SEQ ID NO: 6.

In an additional aspect, the invention provides a method of treating chronic pouchitis in a human subject, said method comprising selecting a human subject who has chronic pouchitis;
administering an initial dose of 300 or 600 mg of an anti-α4β7 antibody, or an antigen binding fragment thereof, to the human subject; administering a second dose of 300 or 600 mg of the anti-α4β7 antibody, or antigen binding fragment thereof, at about two weeks after the initial dose; administering a third dose of 300 or 600 mg of the anti-α4β7 antibody, or antigen
binding fragment thereof, at about six weeks after the initial dose; and subcutaneously administering a dose of 108 mg of the anti-α4β7 antibody, or antigen binding fragment thereof, every one or two weeks after the third dose, wherein the anti-α4β7 antibody, or antigen binding fragment thereof, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

In a further aspect, the invention provides a method of treating chronic pouchitis in a human subject, said method comprising selecting a subject having chronic pouchitis and administering a therapeutically effective dose of an anti-α4β7 antibody to the subject, such that chronic pouchitis is treated, wherein the anti-α4β7 antibody is vedolizumab, wherein the human subject has an endoscopic PDAI subscore of more than 5 at selection and/or the human subject was TNFα naïve at selection.

In one embodiment, the anti-α4β7 antibody, or antigen binding fragment thereof, comprises a heavy chain variable region as set forth in SEQ ID NO: 1 and a light chain variable region as set forth in SEQ ID NO: 5.

In one embodiment, the anti-α4β7 antibody, or antigen binding fragment thereof, comprises a heavy chain as set forth in SEQ ID NO: 9 and a light chain as set forth in SEQ ID NO: 10.

In one embodiment, the therapeutically effective dose is selected from the group consisting of 108 mg, 300 mg, and 600 mg.

In one embodiment, the anti-α4β7 antibody is an IgG antibody, e.g., an IgG1 or an IgG4 isotype.

In one embodiment, the anti-α4β7 antibody, or antigen binding fragment thereof, is humanized.

In one aspect, the invention provides a method of treating chronic pouchitis in a human subject, said method comprising selecting a subject having chronic pouchitis and administering a therapeutically effective dose of an anti-α4β7-antibody to the subject, such that chronic pouchitis is treated, wherein the anti-α4β7 antibody is vedolizumab. In one embodiment, the therapeutically effective dose of vedolizumab is selected from the group consisting of 108 mg, 300 mg, and 600 mg.

In one embodiment, the methods disclosed herein treat patients with moderately to severely active chronic pouchitis, who have undergone proctocolectomy and ileal pouch anal anastomosis for ulcerative colitis.

In one embodiment, the methods disclosed herein treat patients with moderately to severely active chronic pouchitis, who have undergone proctocolectomy and ileal pouch anal anastomosis for ulcerative colitis, and have had an inadequate response with or lost response to antibiotic therapy.

In another aspect, the invention features a method of treating chronic pouchitis in a human subject, said method comprising selecting a human subject who has chronic pouchitis; and administering to the human subject an initial dose of 300 mg of an anti-α4β7-antibody followed by one or more subsequent doses of 300 mg of the anti-α4β7-antibody at least every two weeks, such that the chronic pouchitis is treated in the subject, wherein the anti-α4β7-antibody is vedolizumab.

In still another aspect, the invention provides a method of treating chronic pouchitis in a human subject, said method comprising selecting a human subject who has chronic pouchitis; administering an initial dose of 300 mg of an anti-α4β7-antibody to the human subject: administering a second dose of 300 mg of the anti-α4β7-antibody at about two weeks after the initial dose; administering a third dose of 300 mg of the anti-α4β7-antibody at about six weeks after the initial dose; and administering one or more subsequent doses of 300 mg of the anti-α4β7-antibody every eight weeks after the third dose, wherein the anti-α4β7-antibody is vedolizumab.

In another aspect, the invention features a method of treating chronic pouchitis in a human subject, said method comprising selecting a human subject who has chronic pouchitis; and administering to the human subject an initial dose of 600 mg of an anti-α4β7-antibody followed by one or more subsequent doses of 600 mg of the anti-α4β7-antibody at least every two weeks, such that the chronic pouchitis is treated in the subject, wherein the anti-α4β7-antibody is vedolizumab.

In still another aspect, the invention provides a method of treating chronic pouchitis in a human subject, said method comprising selecting a human subject who has chronic pouchitis; administering an initial dose of 600 mg of an anti-α4β7-antibody to the human subject: administering a second dose of 600 mg of the anti-α4β7-antibody at about two weeks after the initial dose; administering a third dose of 600 mg of the anti-α4β7-antibody at about six weeks after the initial dose; and administering one or more subsequent doses of 300 mg of the anti-α4β7-antibody every eight weeks after the third dose, wherein the anti-α4β7-antibody is vedolizumab.

In certain embodiments, the invention provides a method of treating chronic pouchitis in a human subject, said method comprising selecting a human subject who has chronic pouchitis; and administering to the human subject an initial dose of 300 mg or 600 mg of an anti-α4β7-antibody followed a subsequent dose of 300 mg or 600 mg of the anti-α4β7-antibody at least every two weeks thereafter, such that the chronic pouchitis is treated in the subject, wherein the anti-α4β7-antibody is vedolizumab.

In additional aspects, the invention provides a method of treating chronic pouchitis in a human subject, said method comprising selecting a human subject who has chronic pouchitis;
administering an initial dose of 300 mg or 600 mg of an anti-α4β7-antibody to the human subject; administering a second dose of 300 mg or 600 mg of the anti-α4β7 antibody at about two weeks after the initial dose; administering a third dose of 300 mg or 600 mg of the anti-α4β7-antibody at about six weeks after the initial dose; and administering one or more subsequent doses of 300 mg or 600 mg of the anti-α4β7-antibody every eight weeks after the third dose,
wherein the anti-α4β7-antibody is vedolizumab.

In one embodiment, the initial dose is 300 mg and the subsequent dose is 600 mg and is administered every four weeks or every eight weeks after the initial dose.

In another embodiment, the initial dose is 600 mg and the subsequent dose is 600 mg and is administered every four weeks or every eight weeks after the initial dose.

In another embodiment, the initial dose is 300 mg and the subsequent dose is 300 mg and is administered every four weeks after the initial dose.

In one embodiment, the invention further comprises administering, e.g., on a daily basis, an antibiotic, e.g., ciprofloxacin to the human subject. In one embodiment, the antibiotic is discontinued by 4 weeks following the initial administration of the anti-α4β7 antibody, or antigen binding fragment thereof.

In one embodiment, the human subject received long-term continuous low-dose antibiotic therapy or received frequent pulse antibiotic prior to selection (and used for selection) for the treatment methods described herein.

In one embodiment, the human subject had an ileal pouch anal anastomosis (IPAA) at least one year prior to selection.

In one embodiment, the human subject has an inflammatory bowel disease (IBD). In one embodiment, the IBD is ulcerative colitis, e.g., moderate to severe UC. In one embodiment, the IBD is Crohn's disease, e.g., moderate to severe Crohn's disease.

In one aspect, the methods of the invention are used to achieve clinical remission of pouchitis in a human subject having chronic pouchitis.

In one embodiment, the human subject achieves remission at about 14 weeks following the initial dose of the anti-α4β7 antibody or vedolizumab. In one embodiment, the human subject achieves remission at about 34 weeks following the initial dose of the anti-α4β7 antibody or vedolizumab.

In one embodiment, remission is defined as pouchitis having a modified Pouchitis Disease Activity Index (mPDAI) of <5 and a reduction in overall mPDAI score of ≥2 from baseline.

In one embodiment, the methods disclosed herein are used to treat a human subject having chronic pouchitis, wherein the human subject achieves at least one of the following: symptomatic remission of pouchitis, a change in PDAI endoscopic score at weeks 14 and 34 compared to baseline, a change in PDAI Histologic Findings Score at weeks 14 and 34 compared to baseline, a change in PDAI Score at weeks 14 and 34 compared to baseline, a change in Inflammatory Bowel Disease Questionnaire (IBDQ) Total Score and Subscale Score at weeks 14, 22 and 34 compared to baseline, or a change in 3-Item Cleveland Global Quality of Life (CGQL) at weeks 14, 22 and 34 compared to baseline.

In one embodiment, the anti-α4β7 antibody is administered to the human subject intravenously. In one embodiment, the anti-α4β7 antibody is administered to the human subject subcutaneously.

In one embodiment, the anti-α4β7 antibody used in the methods disclosed herein is etrolizumab. In one embodiment, etrolizumab is subcutaneously administered, for example, to a human subject having chronic pouchitis at a dose of 105 mg at week 0 and every four weeks thereafter.

The invention further provides, in certain embodiments, an anti-α4β7 antibody for use in the treatment of chronic pouchitis in a human subject having inflammatory bowel disease, such as ulcerative colitis. In one embodiment, an anti-α4β7 antibody is vedolizumab and is used in the treatment of chronic pouchitis in a human subject having an inflammatory bowel disease, such as Crohn's disease or ulcerative colitis, by administering 300 mg of vedolizumab to the human patient at week 0, week 2, week 6, and every eight weeks thereafter. In one embodiment, the anti-α4β7 antibody is vedolizumab and is used in the treatment of chronic pouchitis in a human subject having an inflammatory bowel disease, such as Crohn's disease or ulcerative colitis, by administering 300 mg of vedolizumab to the human patient at week 0, week 2, week 6, and every four weeks thereafter. In one embodiment, the anti-α4β7 antibody is vedolizumab and is used in the treatment of chronic pouchitis in a human subject having an inflammatory bowel disease, such as Crohn's disease or ulcerative colitis, by administering 108 mg of vedolizumab to the human patient at week 0, week 2, week 6, and every two weeks thereafter. Vedolizumab may be administered either subcutaneously or intravenously. Also contemplated are other anti-α4β7 antibodies, such as etrolizumab, for use in the treatment of chronic pouchitis using the methods and other treatment parameters (e.g., subpopulations, clinical endpoints) disclosed herein.

### DETAILED DESCRIPTION OF THE INVENTION

More recently, studies have been completed by various parties using vedolizumab to treat chronic pouchitis in patients having ulcerative colitis. In one study described in Bar et al. (Bar et al. (December 2017) Aliment. Pharmacol. Ther. 47:581-587), patients having ulcerative colitis and refractory pouchitis (chronic antibiotic dependent pouchitis) received 300 mg of vedolizumab intravenously at zero, two, six, and ten weeks. At zero, two, six, ten, and fourteen weeks, patients were analyzed for improvement in their refractory pouchitis using the Oresland Score (OS). After fourteen weeks of treatment, all twenty of the patients in the study had a decrease in their OS score (indicating improvement) and seventeen of the patients were able to stop antibiotic treatment. Such a study supports the methods disclosed herein relating to the use of an anti-α4β7 antibody, such as vedolizumab, to treat chronic pouchitis.

### I. Definitions

In order that the present invention may be more readily understood, certain terms are first defined.

As used herein, the term “pouchitis” refers to inflammation of an ileal-pouch anal anastomosis (IPAA) (also referred to herein as an “ileal pouch”). Chronic pouchitis refers to ongoing inflammation of the pouch, which may be subclinical or not and which occasionally flares (recurs). As used herein, the terms “chronic pouchitis” or “recurrent pouchitis” refer to recurring or treatment-refractory disease. In some embodiments, recurrent pouchitis is disease in which flares return after treatment. As used herein, the term “treatment-refractory” refers to a disease or condition which does not generally respond to attempted forms of treatment. In one embodiment, treatment-refractory chronic pouchitis is chronic pouchitis which is non-responsive to antibiotic treatment (i.e., antibiotic-refractory chronic pouchitis). In one embodiment, treatment-refractory chronic pouchitis is antibiotic-dependent, e.g., characterized by long-term antibiotic and/or probiotic therapy. In one embodiment, treatment-refractory chronic pouchitis is chronic pouchitis which is non-responsive to treatment with a TNFα antagonist (i.e., TNF-refractory chronic pouchitis). For a human subject to have pouchitis infers that the subject has undergone a total proctocolectomy and an ileal-pouch anal anastomosis (IPAA).

The term “baseline” refers to a starting point used for a comparison. In one embodiment, a baseline refers to a time point, e.g., day 0, prior to treatment with an anti-α4β7 antibody, or antigen binding fragment thereof.

“Remission”, as it relates to pouchitis, refers to the clinical state wherein a subject who has been diagnosed with pouchitis does not have active disease. In one embodiment, a subject having pouchitis is in remission if the subject has a modified Pouch Disease Activity Index (mPDAI) score less than 5. In another embodiment, remission is defined as a reduction in a subject's overall mPDAI score by ≥2 points from a subject's baseline mPDAI score, e.g., prior to treatment with an anti-α4β7 antibody. In one embodiment, remission is defined as a PDAI score of less than 7 and/or a reduction of 3 or more points in a PDAI score relative to a subject's baseline score prior to treatment.

The term “treatment” or “treating” means any treatment of a disease or disorder in a human subject, including: preventing or protecting against the disease or disorder, that is, causing the clinical symptoms not to develop; inhibiting the disease or disorder, that is, arresting or suppressing the development of clinical symptoms; and/or relieving the disease or disorder that is, causing the regression of clinical symptoms. In one embodiment, treatment of chronic pouchitis is achieved where a subject having chronic pouchitis discontinues antibiotics following administration of a dosing regimen with an anti-α4β7 antibody.

The term “therapeutically effective dose” is defined as an amount sufficient to cure or at least partially arrest the disease and its complications in a patient already suffering from the disease. In one embodiment, a therapeutically effective dose is a dose of an anti-α4β7 antibody that is able to improve a symptom and/or eliminate or reduce complication (e.g., prolonged antibiotic use) associated with chronic pouchitis in a human subject having said disease. In one embodiment, a therapeutically effective dose is a dose of an anti-α4β7 antibody that is able to reduce a Pouchitis Disease Activity Index (PDAI) score or reduce a modified PDAI to a score that is less than that which defined chronic pouchitis in a human subject diagnosed with chronic pouchitis. In another embodiment, a therapeutically effective dose is a dose that is able to discontinue long-term antibiotic or corticosteroid treatment.

The cell surface molecule, “α4β7 integrin,” or “α4β7” (used interchangeably throughout) is a heterodimer of an α4 chain (CD49D, ITGA4) and a β7 chain (ITGB7). Human α4 and β7 genes (GenBank (National Center for Biotechnology Information, Bethesda, Md.) RefSeq Accession numbers NM_000885 and NM_000889, respectively) are expressed by B and T lymphocytes, particularly memory CD4+ lymphocytes. Typical of many integrins, α4β7 can exist in either a resting or activated state. Ligands for α4β7 include vascular cell adhesion molecule (VCAM), fibronectin and mucosal addressin (MAdCAM (e.g., MAdCAM-1)).

As used herein, an “anti-α4β7 antibody” or “anti-α4β7 integrin antibody” refers to an antibody which specifically binds to α4β7 integrin. In one embodiment, an anti-α4β7 antibody blocks or inhibits the binding of α4β7 integrin to one or more of its ligands. In one embodiment, an anti-α4β7 antibody binds to α4β7, but not to α4β1 or αEB7. In one embodiment, an anti-α4β7 antibody is vedolizumab.

The term “antibody” as used herein, means any antigen-binding molecule comprising CDRs that specifically bind to or interact with a particular antigen (e.g., α4β7 integrin). In one embodiment, an antibody is an IgG antibody comprising four polypeptide chains, two heavy (H) chains and two light (L) chains inter-connected by disulfide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. CDRs are defined as the hypervariable domains that determine the antigen binding specificity of an antibody. Examples of IgG antibodies include IgG1, IgG2, IgG3, IgG4. Other types of antibodies comprising heavy and light chains include IgM, IgA1, IgA2, IgD, and IgE.

As used herein, the term “antibody fragment” or “antigen binding fragment”, used interchangeably throughout, of an antibody refers to Fab, Fab′, F(ab′)₂, and Fv fragments, single chain antibodies, functional heavy chain antibodies (nanobodies), as well as any portion of an antibody having specificity toward at least one desired epitope, that competes with the intact antibody for specific binding (e.g., an isolated portion of a complementarity determining region having sufficient framework sequences so as to bind specifically to an epitope). Antigen binding fragments can be produced by recombinant techniques, or by enzymatic or chemical cleavage of an antibody.

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)).

As used herein, the term “humanized antibody” refers to a chimeric antibody that contains minimal sequence derived from non-human immunoglobulins. Generally, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops (complementary determining regions) correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

As used herein, the term “recombinant antibody” refers to an antibody produced as the result of the transcription and translation of a gene carried on a recombinant expression vector. In one embodiment, the vector has been introduced into a host cell. Alternatively, a vector can be used in a cell free system.

As used herein, the term “about” is used synonymously with the term “approximately.” Illustratively, the use of the term “about” indicates that values slightly outside the cited values, namely, plus or minus 5%.

The term “TNFα naïve” or “TNF naïve” refers to the prior treatment history of a human subject having chronic pouchitis, where the human subject was not previously prescribed a TNFα antagonist for treatment of pouchitis and/or ulcerative colitis. Examples of TNFα antagonists include adalimumab, golimumab, etanercept, infliximab, cetrolizumab pegol, or any biosimilars thereof.

### II. Methods for Treating Chronic Pouchitis

The present invention provides methods for the treatment of chronic pouchitis in a human subject, comprising administering an anti-α4β7 integrin antibody, e.g., vedolizumab, or an antigen-binding fragment of an antibody. The treatment methods described herein are used, for example, for inhibiting inflammation of the ileal pouch following proctocolectomy (pouchitis).

Notably, recent studies have shown that an anti-α4β7 integrin antibody can be used to treat chronic pouchitis. As described above, Bar et al. (Bar et al. (December 2017) Aliment. Pharmacol. Ther. 47:581-587), provides results from a study that examined the use of vedolizumab to treat chronic pouchitis. Additional studies supporting the use of an anti-α4β7 integrin antibody, e.g., vedolizumab, for treating chronic pouchitis include 1) Singh et al. (2018) Vedolizumab for Chronic Antibiotic Refractory Pouchitis (Digestive Disease Week (DDW) abstract Sa1829) which describes successful treatment of UC patients having active pouchitis who had failed previous TNF therapy, and who were administered vedolizumab; 2) Kahn et al. (2018) Vedolizumab Treatment in Crohn's Disease of the Pouch (DDW, abstract Su1807) which describes a retrospective analysis of a study examining treatment with vedolizumab of Crohn's patients having active pouchitis who had also failed previous TNF antagonist therapy, where improvements were determined in the treated patients; and 3) Gregory et al. (2018) Vedolizumab for the Treatment of Pouchitis (DDW, abstract Mo1900) which also describes a retrospective review of a Crohn's study examining vedolizumab for treatment of patients having Crohn's and pouchitis, where vedolizumab was able to improve clinical symptoms of pouchitis.

The surgical treatment of choice for patients with colitis, such as ulcerative colitis (UC), is removal of the colon followed by construction of an ileal pouch anal anastomosis (IPAA). Inflammation of the “pouch,” commonly called pouchitis, is the most common long-term complication in these patients and is characterized by watery, sometimes bloody stool associated with urgency, incontinence, abdominal cramps, malaise, and fever. In addition to these abnormalities, biopsy of the pouch shows chronic inflammatory changes with intense infiltration of both acute and chronic inflammatory cells, e.g., polymorphonuclear (PMN) leukocyte (e.g., neutrophil).

Pouchitis is an inflammation of the pouch resulting from restorative proctocolectomy with IPAA used as treatment, for example, in subjects having ulcerative colitis (UC) (including medically refractory UC, UC with dysplasia), and for familial adenomatous polyposis (FAP) or juvenile polyposis coli and Crohn's disease, e.g., Crohn's disease without perianal and/or small bowel disease. The symptoms of pouchitis can include, but are not limited to, increased bowel frequency, urgency, tenesmus, incontinence, nocturnal seepage, rectal bleeding, abdominal cramps, pelvic discomfort, malaise, and fever. While acute pouchitis is generally responsive to antibiotic treatment, a small number of patients (approximately 5% to 19%) who develop acute pouchitis develop chronic pouchitis, which is more difficult to manage (see, for example, Achkar et al. (2005) Clinical Gastroenterology and Hepatology 3:60-66). In some embodiments, the anti-α4β7 antibody can be used to treat chronic pouchitis in a human patient who experienced polyposis, such as familial adenomatous polyposis (FAP) or juvenile polyposis coli. In some embodiments, the anti-α4β7 antibody can be used to treat chronic pouchitis in a human patient who experienced colon cancer.

The Pouchitis Disease Activity Index (PDAI) is a commonly used instrument for grading the severity of pouchitis (described in Sandborn et al. Pouchitis after ileal pouch-anal anastomosis: A pouchitis disease activity index. Mayo Clin Proc 1994; 69:409-15, the contents of which is incorporated herein by reference in its entirety). The PDAI applies quantitative scores to clinical symptoms and endoscopic and histologic acute inflammation. The PDAI was developed as objective and quantitative criteria for pouch inflammation after IPAA. The 18-point overall score is calculated from 3 separate 6-point scales based on clinical symptoms (0 to 6), endoscopic findings (0 to 6) and histologic changes (0 to 6). The PDAI incorporates histologic features of acute inflammation, along with symptom and inflammation on endoscopy, and establishes a cutoff of 7 for differentiation between ‘pouchitis’ (≥7 points) and ‘no pouchitis’ (<7 points). In one embodiment, a human subject for treatment in accordance with the methods disclosed herein is selected for treatment using a PDAI score indicating chronic pouchitis, e.g., a PDAI score of greater than or equal to 7.

In one embodiment, a human subject selected for treatment using the methods disclosed herein has chronic pouchitis and an endoscopic PDAI subscore of 5. In one embodiment, a human subject selected for treatment using the methods disclosed herein has chronic pouchitis and an endoscopic PDAI subscore of 6.

Although the standard PDAI remains a common way to diagnose pouchitis, the modified PDAI, provides an alternative grading instrument to the PDAI. The modified PDAI (mPDAI) includes symptom and endoscopy scores from the PDAI but omits histology scores. The mPDAI offers similar sensitivity and specificity in diagnosing patients with acute or acute relapsing pouchitis. A cutoff of 5 differentiates patients with pouchitis (mPDAI≥5) from patients without pouchitis (mPDAI<5) (Shen et al. Dis Colon Rectum 2003:46(6):748-53, the contents of which is incorporated herein by reference in its entirety). In one embodiment, a human subject for treatment in accordance with the methods disclosed herein is selected for treatment using a mPDAI score indicating chronic pouchitis, e.g., an mPDA score of greater than or equal to 5. In one embodiment, a human subject for treatment in accordance with the methods disclosed herein is selected for treatment using a mPDAI score indicating chronic pouchitis, e.g., an mPDAI score of greater than 5.

The methods of the invention are used to treat a particularly challenging type of pouchitis, i.e., chronic pouchitis. In one aspect, chronic pouchitis is pouchitis that lasts four weeks or more in duration, even in the presence of treatment, e.g., treatment with antibiotics. In another embodiment, chronic pouchitis is pouchitis defined by a modified Pouchitis Disease Activity Index (mPDAI) of ≥5 and greater than two episodes of pouchitis within one year. In another aspect, chronic pouchitis refers to pouchitis that requires long-term continuous low-dose antibiotic therapy (e.g., ciprofloxacin 250-500 mg/day or metronidazole 500 mg/day taken for several weeks or months at a time), or frequent pulse antibiotic therapy.

Prior to treatment with an anti-α4β7 integrin antibody, e.g., vedolizumab, a human subject is selected as having chronic pouchitis (e.g., having a mPDAI score ≥5 and greater than two episodes of pouchitis within the previous year; or requiring long-term continuous low-dose antibiotic therapy, e.g., daily, on an ongoing basis or frequent pulse antibiotic therapy). While acute pouchitis can respond to short-term antibiotic therapy, recurrent or chronic pouchitis can require long-term antibiotic therapy to manage the pouchitis; however, long-term antibiotic use can lead to antibiotic resistance. Some subjects may undertake the more drastic option of surgical removal of the pouch in order to treat chronic or recurrent pouchitis. The methods of the invention provide treatment and/or prevention of recurrent or chronic pouchitis, which is notoriously difficult to treat. As described above, PDAI or mPDAI scores may be used in selecting a human subject for treatment according to the methods disclosed herein.

One advantage of the invention is that, in certain embodiments, the human subject having chronic pouchitis is able to discontinue long term use (also referred to as prolonged use) of a prior therapeutic agent used for the treatment of chronic pouchitis, including, for example, antibiotics, an immunosuppressive agent, an immunomodulator, and/or corticosteroids. Indeed, long term use of a therapeutic agent such as an antibiotic, an immunosuppressive agent, an immunomodulator or a corticosteroid can be associated with detrimental side effects. Thus, an object of the invention is to discontinue or reduce the need for these other agents. Vedolizumab has demonstrated safety for long term use. Long term use of a therapeutic is generally defined as extending in time beyond an accepted treatment regimen or continued use of a therapeutic for treatment where no completion date for the therapeutic is envisioned given the nature of the disease. In one embodiment, long term use of a therapeutic refers to a time period during which the agent is administered (according to standard dosing for the agent), wherein the time period is more than three weeks, at least four weeks, at least two months, at least three months, at least four months, more than four months, at least six months, more than six months, more than eight months, more than twelve months, more than 15 months, more than 18 months, or more than two years or longer. In some embodiments, long term antibiotic use or corticosteroid use is a duration of more than three weeks, more than four weeks, two to six weeks, one to two months or longer. The invention includes treatment of a human subject having chronic pouchitis with an anti-α4β7 antibody where the human subject has been undergoing treatment with a long term therapy, including, but not limited to, an antibiotic, an immunosuppressive agent, an immunomodulator, or a corticosteroid.

In some embodiments, long term refers to the time period during which efficacy of a treatment is maintained. For example, in one embodiment, long-term efficacy or remission of chronic pouchitis is a response, amelioration of at least one symptom or remission lasting at least three months, at least 34 weeks, at least four months, more than four months, at least six months, more than six months, more than eight months, more than twelve months, at least 56 weeks, more than 15 months, more than 18 months, more than two years or longer.

In one embodiment of the invention, an anti-α4β7 antibody, or antigen-binding fragment thereof, e.g., vedolizumab, is administered to a human subject having chronic pouchitis that is associated with IPAA in a subject having ulcerative colitis. In another embodiment of the invention, an anti-α4β7 antibody, or antigen-binding fragment thereof, e.g., vedolizumab, is administered to a human subject having chronic pouchitis that is associated with IPAA in a subject having Crohn's disease. In one embodiment, the human subject is at least 18 years of age. In one embodiment, the human subject is less than 18 years of age. In one embodiment, the human subject is greater than 65 years of age. In one embodiment, the human subject is 5 to 18 or 10 to 15 years of age. In one embodiment, the human subject is an adult.

In another embodiment, an anti-α4β7 antibody, or antigen-binding fragment thereof, is administered in combination with an antibiotic, e.g., ciprofloxacin. The antibiotic treatment can be discontinued following initiation of treatment with an anti-α4β7 antibody, e.g., vedolizumab, or an antigen-binding fragment thereof. In one embodiment, the antibiotic, e.g., ciprofloxacin can be administered at a dosage of, for example, 500 mg, twice daily up to week 4 following the initial administration of the anti-α4β7 antibody, or an antigen-binding fragment thereof.

In one embodiment, the methods of the invention result in remission, e.g., clinically relevant remission, of the pouchitis. For example, the methods of the invention may result in symptomatic remission of pouchitis, reduction in pouch inflammation, symptomatic remission of pouchitis, a change in PDAI endoscopic score at weeks 14 and 34 compared to baseline, a change in PDAI Histologic Findings Score at weeks 14 and 34 compared to baseline, a change in total PDAI Score at weeks 14 and 34 compared to baseline, a change in Inflammatory Bowel Disease Questionnaire (IBDQ) Total Score and Subscale Score at weeks 14, 22 and 34 compared to baseline, and/or a change in 3-Item Cleveland Global Quality of Life (CGQL) at weeks 14, 22 and 34 compared to baseline. Remission may be defined as the subject having a modified Pouchitis Disease Activity Index (mPDAI) of <5 and a reduction in overall mPDAI score of ≥2 from a baseline measure.

Using the methods disclosed herein, remission may be achieved for a given time period, e.g., at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks, at least 32 weeks, at least 33 weeks, at least 34 weeks, at least 35 weeks, at least 36 weeks, at least 5 months, at least 6 months, and so forth. In one embodiment, remission is maintained in a human subject having pouchitis using the methods disclosed herein for over 4 months.

In one embodiment, the human subject selected for treatment may have had a lack of an adequate response with, loss of response to, or was intolerant to treatment with an antibiotic, e.g., ciprofloxacin (Cipro™) or metronidazole (Flagyl™), for the chronic pouchitis. Treatment may allow for a reduction, elimination, or reduction and elimination of antibiotic use by the subject. In one embodiment, the subject may discontinue use of antibiotics following administration of the anti-α4β7 antibody, e.g., vedolizumab. For example, antibiotic use may be discontinued following administration of one, two, three, four, five, or more doses of anti-α4β7 antibody, e.g., vedolizumab. In one embodiment, an anti-α4β7 antibody, e.g., vedolizumab, or an antigen-binding fragment thereof, may be administered to a subject having chronic pouchitis once at weeks 0, 2, 6, 14, 22, and 30, along with antibiotic, e.g., ciprofloxacin, e.g., daily or twice daily up to week 4.

In one embodiment, an anti-α4β7 antibody, e.g., vedolizumab, or antigen-binding fragment thereof, is administered in an effective amount which inhibits binding of α4β7 integrin to a ligand thereof. In some embodiments, an anti-α4β7 antibody, or antigen-binding fragment thereof, inhibits the binding of α4β7 integrin to MAdCAM, or MAdCAM and fibronectin, but not to VCAM. For therapy of pouchitis, an effective amount will be sufficient to achieve the desired therapeutic (including prophylactic) effect (such as an amount sufficient to produce remission, e.g., clinically relevant remission, of pouchitis, symptomatic remission of pouchitis, reduction in pouch inflammation, reduction in PDAI Endoscopic Score, reduction in PDAI Histologic Findings Score, and/or reduction in overall PDAI Score). In some embodiments, an effective amount of an anti-α4β7 antibody, e.g., vedolizumab, or antigen-binding fragment thereof, is an amount sufficient to reduce or eliminate the recurrence of pouchitis, e.g., eliminate relapse of disease.

Treatment of chronic pouchitis with an anti-α4β7 antibody, e.g., vedolizumab, or an antigen-binding fragment thereof, is achieved by administering an effective amount of the antibody to a human subject in need thereof. Exemplary doses of the anti-α4β7 antibody, e.g., vedolizumab, include, but are not limited to, 108 mg, 160 mg, 216 mg, 300 mg, 450 mg or 600 mg.

An anti-α4β7 antibody, e.g., vedolizumab, or an antigen-binding fragment thereof, may be administered to a human subject for treatment according to any method known in the art, e.g., intravenously and/or subcutaneously. In one embodiment, an anti-α4β7 antibody, e.g., vedolizumab, or an antigen-binding fragment thereof, is administered intravenously (IV) to the human subject having chronic pouchitis. In one embodiment, an anti-α4β7 antibody, e.g., vedolizumab, or an antigen-binding fragment thereof, is administered subcutaneously to the human subject having chronic pouchitis. In one embodiment, an anti-α4β7 antibody, e.g., vedolizumab, or an antigen-binding fragment thereof, is administered according to a dosing regimen that includes both intravenous and subcutaneous administration, e.g., an initial dose administered via IV followed by a second dose administered via subcutaneous administration to the human subject having chronic pouchitis. In some embodiments, an anti-α4β7 antibody, e.g., vedolizumab, or an antigen-binding fragment thereof, is administered at 0 and 2 weeks or 0, 2 and 6 weeks via IV followed, e.g., 2, 4, 6 or 8 weeks later, by subsequent doses administered via subcutaneous administration to the human subject having chronic pouchitis. In some embodiments, the subsequent doses administered via subcutaneous administration to the human subject having chronic pouchitis are administered every 1, 2, 3, or 4 weeks. If administered intravenously, the dose of the antibody can be administered to the human subject in about 20 minutes, about 25 minutes, about 30 minutes, about 35 minutes, or about 40 minutes.

In one embodiment, an anti-α4β7 antibody, e.g., vedolizumab, or an antigen-binding fragment thereof, is administered to the human subject having chronic pouchitis at a dose of about 300 mg at 0, 2, and 6 weeks, followed by a 300 mg dose every two weeks thereafter. In one embodiment, an anti-α4β7 antibody, e.g., vedolizumab, or an antigen-binding fragment thereof, is administered to the human subject having chronic pouchitis at a dose of about 300 mg at 0, 2, and 6 weeks, followed by a 108 mg dose every two weeks thereafter. In one embodiment, an anti-α4β7 antibody, e.g., vedolizumab, or an antigen-binding fragment thereof, is administered to the human subject having chronic pouchitis at a dose of about 300 mg at 0 and 2 weeks, followed by a 300 mg dose at 6 weeks and every two weeks thereafter. In one embodiment, an anti-α4β7 antibody, e.g., vedolizumab, or an antigen-binding fragment thereof, is administered to the human subject having chronic pouchitis at a dose of about 300 mg at 0 and 2 weeks, followed by a 300 mg dose at 6 weeks and every four weeks thereafter. In one embodiment, an anti-α4β7 antibody, e.g., vedolizumab, or an antigen-binding fragment thereof, is administered to the human subject having chronic pouchitis at a dose of about 300 mg at 0 and 2 weeks, followed by a 300 mg dose at 6 weeks and every eight weeks thereafter. In one embodiment, an anti-α4β7 antibody, e.g., vedolizumab, or an antigen-binding fragment thereof, is administered to the human subject having chronic pouchitis at a dose of about 300 mg at 0 and 2 weeks, followed by a 300 mg dose at 6 weeks and 10 weeks and/or 14 weeks, followed by a 300 mg dose every eight weeks thereafter. In one embodiment, an anti-α4β7 antibody, e.g., vedolizumab, or an antigen-binding fragment thereof, is administered to the human subject having chronic pouchitis at a dose of about 300 mg at 0 and 2 weeks, followed by a 108 mg dose at 6 weeks and every two weeks thereafter. In one embodiment, an anti-4B7 antibody, e.g., vedolizumab, or an antigen-binding fragment thereof, is administered to the human subject having chronic pouchitis at a dose of about 300 mg at 0 and 2 weeks, followed by a 108 mg dose at 6 weeks and every four weeks thereafter. In one embodiment, an anti-α4β7 antibody, e.g., vedolizumab, or an antigen-binding fragment thereof, is administered to the human subject having chronic pouchitis at a dose of about 300 mg at 0 and 2 weeks, followed by a 108 mg dose at 6 weeks and every one week thereafter. In some embodiments, an anti-α4β7 antibody, e.g., vedolizumab, or an antigen-binding fragment thereof, is administered to the human subject having chronic pouchitis at a dose of about 300 mg at 0 and 2 weeks, followed by a 300 mg dose at 6 weeks and a dose of 108 mg every one, two or four weeks thereafter. In one embodiment, an anti-α4β7 antibody, e.g., vedolizumab, or an antigen-binding fragment thereof, is administered to the human subject having chronic pouchitis at a dose of about 160 mg at 0, 1, 2, 4 and 6 weeks, followed by a 108 mg dose every two weeks thereafter. In this embodiment, all of the doses of an anti-α4β7 antibody may be subcutaneous.

In some embodiments, the 300 mg, 450 mg or 600 mg doses may be IV doses of an anti-α4β7 antibody. In some embodiments, the 108 mg, 160 mg or 216 mg doses of an anti-α4β7 antibody may be administered subcutaneously. For subcutaneous doses, the anti-α4β7 antibody may be self-administered. The longer time periods between doses can be used to maintain remission of symptoms in patients who respond to the treatment. Longer time periods between doses also can be used for smaller, e.g., 50 kg or less or 30 kg or less; or younger patients, e.g., 5-10 years of age. The shorter time periods between doses can be used as a way to increase the amount of therapeutic agent, e.g., if the disease flares or is difficult to treat.

In one aspect, the treatment regimen includes administration of an anti-α4β7 antibody, e.g., vedolizumab, or an antigen-binding fragment thereof, at day 0, administration at about week 2, administration at about week 6 and administration every 4 or 8 weeks thereafter. For example, a treatment regimen for chronic pouchitis can comprise administration, e.g., IV administration, of 300 mg of an anti-α4β7 antibody, e.g., vedolizumab, or an antigen-binding fragment thereof, at day 0, about week 2, about week 6, and every two, four, or eight weeks thereafter.

In one embodiment, a human subject having chronic pouchitis is treated according to the following dosing regimen: an initial dose of 300 mg of an anti-α4β7 antibody, or antigen-binding fragment thereof, followed by a second dose of 300 mg of the anti-α4β7 antibody, or antigen-binding fragment thereof, about two weeks after the initial dose, followed by a third dose of 300 mg of the anti-α4β7 antibody, or antigen-binding fragment thereof, about six weeks after the initial dose; followed by one or more subsequent doses of 300 mg of the anti-α4β7 antibody, or antigen-binding fragment thereof, every eight weeks after the third dose.

In one aspect, the treatment regimen includes administration of a 600 mg dose of an anti-α4β7 antibody, e.g., vedolizumab, or an antigen-binding fragment thereof, at day 0, administration at week 2, administration at week 6 and administration every 4 or 8 weeks thereafter. For example, a treatment regimen for chronic pouchitis can comprise administration, e.g., IV administration, of 600 mg of an anti-α4β7 antibody, e.g., vedolizumab, or an antigen-binding fragment thereof, at day 0, about week 2, about week 6, and every two, four, or eight weeks thereafter.

In one embodiment, a human subject having chronic pouchitis is treated according to the following dosing regimen: an initial dose of 600 mg of an anti-α4β7 antibody, or antigen-binding fragment thereof, (e.g., vedolizumab) followed by a second dose of 600 mg of the anti-α4β7 antibody, or antigen-binding fragment thereof, about two weeks after the initial dose, followed by a third dose of 600 mg of the anti-α4β7 antibody, or antigen-binding fragment thereof, about six weeks after the initial dose; followed by one or more subsequent doses of 600 mg of the anti-α4β7 antibody, or antigen-binding fragment thereof, every eight weeks after the third dose.

In one embodiment, a human subject having chronic pouchitis is treated by subcutaneously administering 105 mg of etrolizumab every four weeks to the subject.

In one embodiment, a human subject having chronic pouchitis is treated by subcutaneously administering 210 mg of etrolizumab every four weeks to the subject.

An anti-α4β7 antibody, e.g., vedolizumab, or antigen-binding fragment thereof, may be administered to a human subject having chronic pouchitis alone or in conjunction with another therapeutic agent. An anti-α4β7 antibody, e.g., vedolizumab, or antigen-binding fragment thereof, used in the methods of the invention can be administered before, along with or subsequent to administration of the additional therapeutic agent, e.g., an antibiotic.

In an embodiment, an anti-α4β7 antibody, e.g., vedolizumab, or an antigen-binding fragment thereof, is co-administered with a medication that is discontinued or decreased over time during the period of treatment with the anti-α4β7 antibody, or antigen-binding fragment thereof. For example, a patient being treated with an antibiotic (e.g. ciprofloxacin, metronidazole) at the beginning, or prior to, treating with an anti-α4β7 antibody, or an antigen-binding fragment thereof, would undergo a regimen of administration for a period of time concomitant with the anti-α4β7 antibody, or the antigen-binding fragment thereof, and subsequently reduce or discontinue to antibiotic. For example, an anti-α4β7 antibody, or an antigen-binding fragment thereof, can be administered once at weeks 0, 2, 6, along with daily administration of the antibiotic beginning at or prior to week 0. The antibiotic can be decreased in amount or discontinued following one, two, three, four, or five weeks of daily administration, e.g., the antibiotic can be discontinued at week 4. In one embodiment, the antibiotic, e.g., ciprofloxacin, is administered as a 500 mg tablet, orally twice daily, up to week 4.

In certain embodiments, the human subject who is treated with the therapeutic methods described herein has chronic pouchitis but does not have one or more of the following gastrointestinal characteristics: Crohn's disease (CD), or CD of the pouch; irritable pouch syndrome (IPS); cuffitis; mechanical complications of the pouch (e.g., pouch stricture or pouch fistula); or requires or has a planned surgical intervention for UC during the planned treatment.

In certain embodiments, the human subject who is treated with the therapeutic methods described herein has chronic pouchitis but does not have one or more of the following infectious disease characteristics: evidence of an active infection (e.g., sepsis, cytomegalovirus, or listeriosis) during selection for treatment or baseline; active or latent tuberculosis (TB), regardless of treatment history (as evidenced by any of the following: history of TB; a diagnostic TB test performed during selection or baseline that is positive, as defined by, for example, a positive QUANTIFERON® (Cellectis Limited, Chadstone, Victoria) test or 2 successive indeterminate QUANTIFERON tests or a tuberculin skin test reaction ≥10 mm (≥5 mm in subjects receiving the equivalent of >15 mg/day prednisone; or a chest X-ray within 3 months prior to Week 0 of the treatment, which is suspicious for pulmonary TB, and a positive or 2 successive indeterminate QUANTIFERON test within 30 days prior to selection or baseline or during the Screening Period); or a positive test result for hepatitis B surface antigen (HBsAg), antibody to hepatitis C virus (anti-HCV), at selection or baseline or a known history of human immunodeficiency virus infection (e.g., common variable immunodeficiency, human immunodeficiency virus [HIV] infection, organ transplantation). In certain embodiments, the human subject who is treated with the therapeutic methods described herein has chronic pouchitis but does not have one or more of the following characteristics: prior exposure to vedolizumab, natalizumab, efalizumab, rituximab, etrolizumab, or anti-mucosal addressin cell adhesion molecule-1 (MAdCAM-1) therapy; a history of hypersensitivity or allergies to vedolizumab or its components; allergies to and/or contraindications for ciprofloxacin (including interacting drugs such as tizanidine); has received an investigational or approved biologic or biosimilar agent within 60 days or 5 half-lives of selection (whichever is longer); has received an investigational nonbiologic therapy within 30 or 5 half-lives days prior to selection or baseline (whichever is longer); has received an approved nonbiologic therapy (including 5-aminosalicylate [5-ASA], corticosteroid, azathioprine, 6-mercaptopurine [6-MP], etc.) in an investigational protocol within 30 days or 5 half-lives prior to selection or baseline (whichever is longer); has received any live vaccinations within 30 days prior to selection or baseline; has a positive progressive multifocal leukoencephalopathy (PML) subjective symptom checklist at selection or baseline; has a history of tendon rupture disorders related to quinolone administration; has had a kidney, heart, or lung transplant; has myasthenia gravis, peripheral neuropathy, QT prolongation, or a history of seizure; has a history of malignancy (except for the following: adequately-treated non-metastatic basal cell skin cancer; squamous cell skin cancer that has been adequately treated and that has not recurred for at least 1 year prior to the selection visit or baseline; and history of cervical carcinoma in situ that has been adequately treated and that has not recurred for at least 3 years prior to selection or baseline;). In certain embodiments, the human subject who is treated with the therapeutic methods described herein has chronic pouchitis but does not have one or more of the following: a history of a major neurological disorders, including stroke, multiple sclerosis, brain tumor, demyelinating, or neurodegenerative disease; and/or the following laboratory abnormalities during selection or baseline: hemoglobin level <8 g/dL; white blood cell (WBC) count <3×10\hat{ }9/L; lymphocyte count <0.5×10\hat{ }9/L; platelet count <100×10\hat{ }9/L or ≥1200×10\hat{ }9/L; alanine aminotransferase (ALT) or aspartate aminotransferase (AST)>3×the upper limit of normal (ULN); alkaline phosphatase >3×ULN; or serum creatinine >2×ULN; has glucose-6-phosphate dehydrogenase (G6PD) deficiency.

In certain embodiments, the human subject who is treated with the therapeutic methods described herein has chronic pouchitis but does not have one or more of the following gastrointestinal characteristics: chronic hepatitis B virus (HPV) infection confirmed by antiboduies to either surface or core proteins and a positive polymerase chain reaction for HPV DNA; chronic hepatitis C virus infection confirmed by a viral load test; active infection with Clostridium difficile during screening, e.g., as confirmed by a laboratory test; use of tizanidine, methotrexate and/or zolpidem from Day 1 of treatment to week 4; continuous use for 2 weeks of non-steroidal anti-inflammatory drug within 30 days prior to randomization and through the study until week 34, except daily low dose (e.g., 100 mg) acetylsalicylic acid for cardiovascular prophylaxis; use of rectal products, such as enemas or suppositories, e.g., 5-ASA or corticosteroid, within 15 days prior to randomization (e.g., day 1) and through week 34.

In certain embodiments, the human subject who is treated with the therapeutic methods described herein is permitted the use of one or more of the following medications during the study: a drug metabolized by cytochrome P450 1A2 enzyme (CYP1A2), e.g., with caution or monitoring, during ciproflaxin administration; additional antibiotics after week 14, e.g., as needed for flare of disease; oral 5-ASA, if dose is stable for at least 2 weeks prior to randomization and through week 34; antibiotic therapy for pouchitis, e.g., additional to ciproflaxin, if stable dose at least 2 weeks prior to randomization; oral corticosteroid therapy for pouchitis if stable dose at least 4 weeks prior to randomization, but tapered after week 4 of the study; probiotic therapy, e.g., Saccharomyces boulardii, and/or immunomodulator, such as azathioprine, or 6-mercaptorpurine, if stable dose at least 8 weeks prior to randomization and through week 34.

In certain embodiments, the human subject who is treated with the therapeutic methods described herein has one or more of the following conditions, e.g., myasthenia gravis; peripheral neuropathy; QT prolongation; and/or a history of seizure.

In certain embodiments, during the methods of the invention, oral corticosteroid use for treatment of chronic pouchitis is tapered to be reduced or discontinued, e.g., by week 7, 8, 9 or 10. In some embodiments, the maximum dose of prednisone is 20 or 30 mg/day, and is tapered, e.g., to 10 mg/day, 5 mg/day or discontinued. In some embodiments, the maximum dose of budesonide is 9 mg/day, and is tapered, e.g, to discontinuation, e.g., in 3 mg/day intervals. In some embodiments, the maximum dose of beclomethasone disproportionate, or equivalent, is 5 mg/day, and is tapered, e.g., to discontinuation.

In certain embodiments, the methods of the invention include the treatment of a human subject who has failed, been non-responsive to, and/or has had an inadequate response to a tumor necrosis factor (TNF) antagonist (e.g., adalimumab, infliximab, golimumab, etanercept, and/or certolizumab pegol (e.g., CIMZIA®). The treatment methods disclosed herein may be used to treat these hard-to-treat patients.

Further, the methods of the invention disclosed herein may also be used to treat a subject who has chronic pouchitis and who is TNF naïve, in that the subject has not had previous TNF antagonist therapy for the treatment of chronic pouchitis or IBD, such as ulcerative colitis.

In certain embodiments, the methods of the invention include the treatment of a human subject who has failed corticosteroid treatment.

In some embodiments, the methods of the invention disclosed herein include treating a human subject with chronic pouchitis who also uses nicotine or smokes cigarettes. In some embodiments, the methods of the invention disclosed herein include treating a human subject with pouchitis who also has a neurodegenerative disease. In some embodiments, the methods of the invention disclosed herein include treating a human subject with chronic pouchitis who also has arthritis. In some embodiments, the methods of the invention disclosed herein include treating a human subject having chronic pouchitis who also has diabetes or heart disease. In some embodiments, the methods of the invention disclosed herein include treating a human subject having chronic pouchitis who also has backwash ileitis. In some embodiments, the methods of the invention disclosed herein include treating a human subject having chronic pouchitis who also has inflamed and hardened bile ducts in the liver.

The treatment methods disclosed herein may be used to treat adult patients with chronic pouchitis who have had an inadequate response with, lost response to, or were intolerant to a tumor necrosis factor (TNF) blocker or immunomodulator; or had an inadequate response with, were intolerant to, or demonstrated dependence on corticosteroids.

In some embodiments, the methods described herein may be used to treat a human patient with chronic pouchitis wherein the patient is administered an additional therapeutic agent or agents in combination. By combination, it is intended to mean that the additional agent or agents are administered before, concurrently with, or following treatment with the anti-α4β7 antibody. Combination therapy is not intended to mean a composition comprising both the anti-α4β7 antibody and the additional agent(s). For example, the human patient with chronic pouchitis is administered a combination therapy comprising an anti-α4β7 antibody, an antibiotic and an oral corticosteroid. In another example, the human patient with chronic pouchitis is administered a combination therapy comprising an anti-α4β7 antibody, an antibiotic, an oral corticosteroid and a TNF antagonist. In another example, the human patient with chronic pouchitis is administered a combination therapy comprising an anti-α4β7 antibody, an antibiotic and a TNF antagonist. In another example, the human patient with chronic pouchitis is administered a combination therapy comprising an anti-α4β7 antibody, an antibiotic, an oral corticosteroid and an immunomodulator. In another example, the human patient with chronic pouchitis is administered a combination therapy comprising an anti-α4β7 antibody, an antibiotic and an immunomodulator.

In the foregoing embodiments, one or more of the agents may be discontinued during treatment with the anti-α4β7 antibody. The methods of the invention may, in certain embodiments, provide for the discontinuation of other therapeutic agents used for long term therapy, e.g., antibiotics or corticosteroids. Discontinuation of such agents is beneficial to the human subject as it may decrease the number of medication-related side effects, may lower the cost of treatment, may result in better patient compliance, and may improve the subject's overall quality of life. In some embodiments, e.g., during a relapse, a discontinued agent may be re-introduced, e.g., for two to 6 weeks, to restore the response or remission. In certain embodiments, after evidence of remission, e.g., remission with the multiple agents, one or more of the agents may be discontinued. In some embodiments, the anti-α4β7 antibody is administered as a single therapeutic agent for the treatment of chronic pouchitis during long-term remission of the chronic pouchitis.

The anti-α4β7 antibody can be administered to the individual as part of a pharmaceutical or physiological composition for the treatment of chronic pouchitis. Such a composition can comprise an anti-α4β7 antibody as described herein, and a pharmaceutically or physiologically acceptable carrier. In certain embodiments, a pharmaceutical or physiological composition for co-therapy comprises an anti-α4β7 antibody and one or more additional therapeutic agents. In some embodiments, an anti-α4β7 antibody and an additional therapeutic agent are components of separate compositions which are be mixed together prior to administration or administered separately. Formulations will vary according to the route of administration selected (e.g., solution, emulsion, capsule). Suitable carriers may contain inert ingredients which do not interact with the immunoglobulin or antigen-binding fragment and/or additional therapeutic agent. Standard pharmaceutical formulation techniques may be employed, such as those described in Remington's Pharmaceutical Sciences, Mack Publishing Company, Easton, Pa. Suitable carriers for parenteral administration include, for example, sterile water, physiological saline, bacteriostatic saline (saline containing about 0.9% (9 mg/ml) benzyl alcohol), phosphate-buffered saline, 5% dextrose, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986). For inhalation, the agent may be solubilized and loaded into a suitable dispenser for administration (e.g., an atomizer, nebulizer or pressurized aerosol dispenser).

Examples of pharmaceutical formulations suitable for administering an anti-α4β7 antibody, e.g., vedolizumab, are described in US Patent Application Pub. Nos. US 20140341885 and US 20140377251, both of which are incorporated by reference herein.

### II. Anti-α4β7 Antibodies for Use in Treatment Methods

The treatment methods disclosed herein are based on the administration of an anti-α4β7 antibody, or an antigen binding fragment thereof, to a human subject having chronic pouchitis. In a particular embodiment, the anti-α4β7 antibody is vedolizumab or an anti-α4β7 antibody or antigen binding fragment having the binding regions, e.g., CDRs, corresponding to vedolizumab.

The α4β7 integrin is expressed on the surface of a discrete subset of lymphocytes that preferentially migrate into the gastrointestinal tract. MAdCAM-1 is mainly expressed on gut endothelial cells or secondary lymphoid regions, such as Peyer's patches, and plays a critical role in the homing of T-lymphocytes to gut lymph tissue. The interaction of the α4β7 integrin with MAdCAM-1 has been implicated as an important contributor to the chronic inflammation that is a hallmark of ulcerative colitis and Crohn's disease.

Vedolizumab is a humanized monoclonal antibody that specifically binds to the α4β7 integrin and blocks the interaction of α4β7 integrin with mucosal addressin cell adhesion molecule-1 (MAdCAM-1) and inhibits the migration of memory T-lymphocytes across the endothelium into inflamed gastrointestinal parenchymal tissue. Vedolizumab does not bind to or inhibit function of the α4β1 and αEβ7 integrins and does not antagonize the interaction of α4 integrins with vascular cell adhesion molecule-1 (VCAM-1).

Vedolizumab is effective for induction and maintenance therapy of ulcerative colitis (UC) and Crohn's disease. Vedolizumab abrogates the interaction of α4β7 integrin on memory T and B cells with MAdCAM-1 expressed on the vascular endothelium in the gut. MAdCAM-1 levels are elevated in IBD. As described herein, vedolizumab is effective in the treatment of other chronic inflammatory diseases of the GI tract, including pouchitis.

Vedolizumab is also known by its trade name ENTYVIO® (Takeda Pharmaceuticals, Inc.). Vedolizumab is a humanized IgG1 monoclonal antibody. The sequences of vedolizumab are described in US Patent Application Pub. Nos. US 20140341885 and US 20140377251, incorporated by reference herein.

The heavy chain variable region of vedolizumab is provided herein as SEQ ID NO: 1, and the light chain variable region of vedolizumab is provided herein as SEQ ID NO:5. Vedolizumab comprises a heavy chain variable region comprising a CDR1 of SEQ ID NO:2, a CDR2 of SEQ ID NO:3, and a CDR3 of SEQ ID NO:4. Vedolizumab comprises a light chain variable region comprising a CDR1 of SEQ ID NO:6, a CDR2 of SEQ ID NO:7 and CDR3 of SEQ ID NO:8. Vedolizumab and the sequences of vedolizumab are also described in U.S. Patent Publication No. 2014/0341885 and U.S. Patent Publication No. 2014/0377251, the entire contents of each which are expressly incorporated herein by reference in their entireties. In one embodiment, the anti-α4β7 antibody used in the methods herein comprises a heavy chain comprising an amino acid sequence as set forth in SEQ ID NO: 9 and comprises a light chain comprising an amino acid sequence as set forth in SEQ ID NO: 10.

Anti-α4β7 antibodies, particularly vedolizumab or an antibody or antigen binding fragment having the binding regions, i.e., CDRs or variable regions, of vedolizumab, are useful in the methods of the invention for the treatment of chronic pouchitis.

It should be noted that where antibodies are described herein, antigen binding fragments may also be used.

Also included in the invention is the use of the methods disclosed herein with an alternative antibody. Specifically, the methods described herein may be performed using an antibody, or an antigen binding fragment thereof, that binds to α4 integrin, including, but not limited to natalizumab. In another alternative, the methods described herein may be performed using an antibody, or an antigen binding fragment thereof, that binds to β7 integrin, including, but not limited to etrolizumab which binds to both integrins α4β7 and αEβ7 (sequences of etrolizumab are described in US 20180086833, which is incorporated by reference herein).

In another alternative, the methods described herein may be performed using an antibody, or an antigen binding fragment thereof, that binds to MAdCAM-1, including, but not limited to, PF-00547659, a fully human monoclonal antibody. In another alternative, the methods described herein may be performed using an antibody, or an antigen binding fragment thereof, that binds to α4β7 integrin, including, but not limited to AMG-181, a fully human monoclonal antibody.

The following example exemplifies methods for treating pouchitis in a human subject.

### Example: Study of Efficacy and Safety of Vedolizumab in the Treatment of Chronic Pouchitis in Human Patients

This example describes a phase 4, randomized, double-blind, placebo-controlled, multicenter study to evaluate the efficacy and safety of vedolizumab intravenous (IV) 300 mg over a 34-week treatment period (with the last dose at week 30) in subjects with a proctocolectomy and ileal pouch anal anastomosis (IPAA) for ulcerative colitis (UC) who developed chronic or recurrent pouchitis.

### Overview

Vedolizumab was tested to treat human subjects who have chronic pouchitis. This study investigated the healing of inflammation of ileal pouch in subjects who take vedolizumab. The purpose of this study was to compare the efficacy of vedolizumab intravenous (IV) versus placebo. The study evaluated, e.g., the percentage of participants with chronic or recurrent pouchitis who achieved clinically relevant remission in each group.

The study (Clinical Trial Identifier No. NCT02790138 (the EARNEST study)) enrolled 102 adult subjects having chronic or recurrent pouchitis. Patients received either placebo IV or vedolizumab IV at a dose of 300 mg.

One aspect of the inclusion criteria, was that the patient had pouchitis that was recurrent or chronic. Chronic or recurrent pouchitis is defined as a modified Pouchitis Disease Activity Index (mPDAI) score of 5 or more assessed as the average from 3 days immediately prior to the baseline endoscopy and a minimum endoscopic subscore of 2 (outside the staple or suture line) with either a) 3 recurrent episodes in 1 year prior to the screening visit, each treated with 2 or more weeks of antibiotic or other prescription therapy, or b) requiring maintenance antibiotic therapy taken continuously for 4 weeks immediately prior to the baseline endoscopy visit.

Endoscopy was performed at screening (baseline endoscopy), week 14, and week 34.

Subjects were randomly assigned to one of the two treatment groups which remained undisclosed to the patient and study doctor during the study (unless there was an urgent medical need). Treatment group 1 was administered vedolizumab 300 mg IV. Treatment group 2 was administered a placebo.

All subjects in group 1 of the study received an intravenous (IV) infusion of 300 mg vedolizumab at week 0, i.e., day 1, weeks 2, 6, 14, 22, and 30, along with concomitant antibiotic treatment with ciprofloxacin 500 mg twice daily, orally, through week 4 (see Table 1, below). Participants also participated in a long-term follow-up, by phone after the last dose of study drug up to Week 56.

This multicenter trial was conducted in North America and Europe. The overall time to participate in this study was 34 weeks. Subjects made multiple visits to the clinic, plus a visit 18 weeks after the last dose of study drug for a safety follow-up assessment. Subjects also participated in a long-term safety follow-up, by phone, at 6 months after the last dose of study drug (Week 56).

TABLE 1
Study Arms

| | |
|---|---|
| Experimental: Vedolizumab 300 mg | Drug: Vedolizumab |
| Vedolizumab 300 mg, intravenous (IV) infusion, once at Day 1, Weeks, | Vedolizumab IV infusion |
| 2, 6, 14, 22, and 30 along with ciprofloxacin 500 mg, tablet, orally | Other Names: |
| twice daily up to week 4. Week 0 represents Day 1 of treatment. | Entyvio |
| | MLN0002 IV |
| | Kynteles |
| | Drug: Ciprofloxacin |
| | Ciprofloxacin Tablets |
| Placebo Comparator: Placebo | Drug: Ciprofloxacin |
| Vedolizumab placebo-matching IV infusion, once at Day 1, Weeks 2, | Ciprofloxacin Tablets |
| 6, 14, 22, and 30 along with ciprofloxacin 500 mg, tablet, orally twice | Drug: Vedolizumab Placebo |
| daily up to week 4. | Vedolizumab placebo-matching IV |
| | infusion |

### Eligibility

The age of eligibility for the study was 18 years to 80 years old. All sexes were eligible. Healthy volunteers were not eligible.

### Inclusion Criteria

Subject eligibility was determined according to the following criteria prior to entry into the study:

The subject had a history of ileal pouch anal anastomosis (IPAA) for ulcerative colitis (UC) completed at least 1 year prior to the Day 1 of the study.

The subject had pouchitis that is chronic or recurrent, defined by a modified Pouchitis Disease Activity Index (mPDAI)≥5 and >2 episodes within 1 year of the Screening Visit or requiring long-term continuous low-dose antibiotic therapy taken daily on an ongoing basis (e.g., ciprofloxacin 250-500 mg/day or metronidazole 500 mg/day taken for several weeks or months at a time) or frequent pulse antibiotic therapy. An alternative criteria was that the subject had a mPDAI score of 5 or more assessed as the average from 3 days immediately prior to the baseline endoscopy and a minimum endoscopic subscore of 2 (outside the staple or suture line) with either a) 3 recurrent episodes within 1 year prior to the screening visit, each treated with 2 weeks of antibiotic or other prescription therapy or b) requiring maintenance antibiotic therapy taken continuously for 4 weeks immediately prior to the baseline endoscopy visit.

Each subject took ciprofloxacin (500 mg twice daily) on Day 1 and through Week 4, regardless of the previous treatment and stopped any previous antibiotic therapy on Day 1 of the study (additional courses of antibiotics were to be allowed, as needed, for flares after Week 14).

A male subject who was nonsterilized and sexually active with a female partner of childbearing potential agreed to use adequate contraception from signing of informed consent throughout the duration of the study and for 18 weeks after last dose.

A female subject of childbearing potential who was sexually active with a nonsterilized male partner agreed to use routinely adequate contraception from signing of informed consent throughout the duration of the study and for 18 weeks after last dose.

### Exclusion Criteria

Certain human subjects were excluded for the purposes of the clinical trial. Generally, the exclusion criteria were divided into 3 categories: gastrointestinal exclusion criteria, infectious disease exclusion criteria, and general exclusion criteria. If the subject was female, the subject was excluded if pregnant or lactating or intending to become pregnant or nurse before, during, or within 18 weeks after the last dose of study medication; or intending to donate ova during such time period. If male, the subject was excluded from the study if the subject intended to donate sperm or father a child during the course of this study or for 18 weeks after the last dose of study medication. The subject was also excluded if the subject has a history of drug abuse (defined as any illicit drug use) or a history of alcohol abuse within 1 year prior to the Visit 1.

A subject was excluded from the study if the subject had Crohn's disease (CD), CD of the pouch, irritable pouch syndrome (IPS), isolated or predominant cuffitis, diverting stoma, or mechanical complications of the pouch. Subjects were also excluded if they had previous treatment with vedolizumab, natalizumab, efalizumab, rituximab, etrolizumab, or anti-mucosal addressin cell adhesion molecule-1 (MAdCAM-1) therapy. Subjects were also excluded if they have received any investigational or approved biologic or biosimilar agent within 60 days of randomization.

The maximum dose of oral corticosteroids for the treatment of pouchitis that would be co-administered with vedolizumab IV was 20 mg/day prednisone or 9 mg/day budesonide or 5 mg/day beclomethasone dipropionate (or equivalent) as long as they had been used at a stable dose for at least 4 weeks prior to randomization. It was required that subjects receiving oral corticosteroids begin a tapering regimen by Week 4 (Visit 4) of the study. The recommended tapering schedule was to be finished by Week 8, if possible.

Exclusion criteria are further summarized below:

### Gastrointestinal Exclusion Criteria

1. Had Crohn's disease (CD), or CD of the pouch.
2. Had irritable pouch syndrome (IPS).
3. Had isolated or predominant cuffitis.
4. Had mechanical complications of the pouch (e.g., pouch stricture or pouch fistula).
5. Currently required or had a planned surgical intervention for UC during the study.
6. Had diverting stoma.

### Infectious Disease Exclusion Criteria

1. Had evidence of an active infection (e.g., sepsis, cytomegalovirus, or listeriosis) during Screening.
2. Had active or latent tuberculosis (TB), regardless of treatment history, as evidenced by any of the following:
   1. A diagnostic TB test performed within 30 days of Screening or during the Screening Period that was positive, as defined by:
      a. A positive QuantiFERON test or 2 successive indeterminate QuantiFERON tests. OR
      b. A tuberculin skin test reaction ≥10 mm (≥5 mm in participants receiving the equivalent of ≥15 mg/day prednisone).
      OR
   2. Chest X-ray within 3 months prior to Day 1 that was suspicious for pulmonary TB, and a positive or 2 successive indeterminate QuantiFERON test within 30 days prior to Screening or during the Screening Period.
   3. Had chronic hepatitis B virus (HBV) infection* or chronic hepatitis C virus (HCV) infection** or a known history of human immunodeficiency virus (HIV) infection (or was found to be seropositive at Screening) or participant was immunodeficient (e.g., due to organ transplantation, history of common variable immunodeficiency, etc.).
      * Participants who were positive for hepatitis B virus surface antigen (HBsAg) were excluded. For participants who were negative for HBsAg but were positive for either surface antibodies and/or core antibodies, HBV Deoxyribonucleic acid (DNA) polymerase chain reaction was performed and if any test result met or exceeded detection sensitivity, the participant was excluded.
      ** If participant was HCV antibody positive, then a viral load test was performed. If the viral load test was positive then the participant was excluded.
   4. Had evidence of active infection with Clostridium (C) difficile during Screening (was to be confirmed by laboratory test)

### General Exclusion Criteria

a. Had any prior exposure to vedolizumab, natalizumab, efalizumab, rituximab, etrolizumab, or anti-mucosal addressin cell adhesion molecule-1 (MAdCAM-1) therapy.
b. Had a history of hypersensitivity or allergies to vedolizumab or its components.
c. Had allergies to and/or contraindications for ciprofloxacin, a history of tendon disorders related to quinolone administration and/or glucose-6-phosphate dehydrogenase (G6PD) deficiency. Further conditions requiring precautions for use of ciprofloxacin were to be considered based on local prescribing information.
d. Was taking, had taken, or was required to take any excluded medications.
e. Had received any investigational or approved biologic or biosimilar agent within 60 days prior to Randomization.
f. Had received an investigational nonbiologic therapy within 30 days prior to Randomization.
g. Had received an approved nonbiologic therapy (including 5-aminosalicylate [5-ASA], corticosteroid, azathioprine, 6-mercaptopurine [6-MP], etc.) in an investigational protocol within 30 days prior to Randomization.
h. Had received any live vaccinations within 30 days prior to randomization.
i. Had a positive progressive multifocal leukoencephalopathy (PML) subjective symptom checklist at Screening.
j. Had a kidney, heart, or lung transplant.
k. Had a history of malignancy, except for the following: adequately-treated non-metastatic basal cell skin cancer; squamous cell skin cancer that had been adequately treated and that had not recurred for at least 1 year prior to the Screening visit; and history of cervical carcinoma in situ that had been adequately treated and that had not recurred for at least 3 years prior to Screening. Participants with a remote history of malignancy (e.g., >10 years since completion of curative therapy without recurrence) were considered based on the nature of the malignancy and the therapy received and must have been discussed with the sponsor on a case-by-case basis prior to enrollment.
l. Had a history of any major neurological disorders, including stroke, multiple sclerosis, brain tumor, demyelinating, or neurodegenerative disease.
m. Had conditions, which in the opinion of the investigator, may interfere with the participant's ability to comply with the study procedures.
n. Had any unstable or uncontrolled cardiovascular, pulmonary, hepatic, renal, gastrointestinal (GI), genitourinary, hematological, coagulation, immunological, endocrine/metabolic, neurologic, or other medical disorder that, in the opinion of the investigator, would confound the study results or compromise participant safety.
o. Had any of the following laboratory abnormalities during the Screening Period:
   a. Hemoglobin level <8 g/dL.
   b. White blood cell (WBC) count <3×10\hat{ }9/L.
   c. Lymphocyte count <0.5×10\hat{ }9/L.
   d. Platelet count <100×10\hat{ }9/L or ≥1200×10\hat{ }9/L.
   e. Alanine aminotransferase (ALT) or aspartate aminotransferase (AST)≥3×the upper limit of normal (ULN).
   f. Alkaline phosphatase ≥3×ULN.
   g. Serum creatinine ≥2×ULN.
p. If female, the participant was pregnant or lactating or intending to become pregnant or nurse before, during, or within 18 weeks after the last dose of study medication; or intending to donate ova during such time period.
q. If male, the participant intended to donate sperm or father a child during the course of this study or for 18 weeks after the last dose of study medication.
r. Was an immediate family member, study site employee, or was in a dependent relationship with a study site employee who was involved in conduct of this study (e.g., spouse, parent, child, sibling) or may consent under duress.
s. Had a history of drug abuse (defined as any illicit drug use) or a history of alcohol abuse within 1 year prior to Screening.

### Primary Outcome Measures

The primary efficacy outcome measure was the percentage of subjects with chronic or recurrent pouchitis achieving clinically relevant remission after 14 weeks of treatment. Clinically relevant remission was defined as an mPDAI score of <5 and a reduction in overall score by 2 points from baseline. The 12-point modified Pouchitis Disease Activity Index (mPDAI) score was calculated as a sum of from two 6-point subscales, where 0=best and 12=worst: 1) Clinical Symptoms: Stool Frequency (0=usual to postoperative stool frequency to 2=three or more stools/day>postoperative usual); Rectal bleeding (0=None or rare to 1=Present daily); Fecal urgency or abdominal cramps (0=None to 2=Usual), Fever [temperature >37.8 degrees C.] (0=Absent and 1=Present) for a clinical symptoms subscore of 0 (best) to 6 (worst); 2) Endoscopic Inflammation Findings: Edema, Granularity, Friability, Loss of vascular pattern, Mucous exudates and Ulcerations. Each item was scored on a scale of 0=not present to 1=present summed up to an endoscopic subscore ranging from 0 (best) to 6 (worst) where higher scores indicated more severe disease.

### Secondary Outcome Measures

The secondary outcome measures were as follows:
1. Change in modified Pouchitis Disease Activity Index (mPDAI). Percentage of subjects achieving mPDAI<5 and a reduction of overall score by at least 2 points from baseline after 34 weeks of treatment (where the last dose is at 30 weeks).

Clinically relevant remission was defined as mPDAI score <5 and a reduction of mPDAI score by ≥2 points from Baseline. The 12-point mPDAI score was calculated as a sum of from two 6-point subscales, where 0=best and 12=worst: 1) Clinical Symptoms: Stool Frequency (0=usual to postoperative stool frequency to 2=3 or more stools/day>postoperative usual); Rectal bleeding (0=None or rare to 1=Present daily); Fecal urgency or abdominal cramps (0=None to 2=Usual), Fever [temperature >37.8 degrees C.] (0=Absent and 1=Present) for a clinical symptoms subscore of 0 (best) to 6 (worst); 2) Endoscopic Inflammation Findings: Edema, Granularity, Friability, Loss of vascular pattern, Mucous exudates and Ulcerations. Each item was scored on a scale of 0=not present to 1=present summed up to an endoscopic subscore ranging from 0 (best) to 6 (worst) where higher scores indicated more severe disease.
2. Change in Pouchitis Disease Activity Index (PDAI). Percentage of subjects achieving PDAI score <7 and a reduction of overall score by at least 3 points from baseline PDAI score after 14 weeks of treatment and after 34 weeks of treatment (where the last dose is at 30 weeks).

PDAI remission is PDAI score <7 and a reduction of PDAI score by ≥3 points from Baseline. The 18-point PDAI score was calculated as a sum of three 6-point scales with total possible subscore of 0 (best) to 6 (worst) and possible total score of 0 (best) to 18 (worst): 1) Clinical Symptoms: Stool Frequency (0=usual to postoperative stool frequency to 2=3 or more stools/day>postoperative usual); Rectal bleeding (0=None or rare, 1=Present daily); Fecal urgency/abdominal cramps (0=None to 2=Usual), Fever [temperature>37.8 degrees C.] (0=Absent, 1=Present); 2) Endoscopic Inflammation: Edema, Granularity, Friability, Loss of vascular pattern, Mucous exudates, Ulcerations. Each item was scored on scale of 0=not present to 1=present summed up to an endoscopic subscore ranging from 0 (best) to 6 (worst); 3) Acute Histologic Inflammation: Polymorphic nuclear leukocyte infiltration (0=None to 3=Severe plus crypt abscess), Ulceration per low power field [mean] (0=0% to 3=>50%) where higher scores indicated more severe disease.
3. Time to PDAI Remission. (Time Frame: Baseline up to Week 34). Remission was defined as a PDAI score <7 and a decrease in PDAI score of at least 3 points from baseline.

Time to remission-time in days from start of treatment to PDAI Remission (PDAI score <7 and decrease in PDAI score ≥3 points from Baseline). 18-point PDAI score-calculated as sum of 3, 6-point scales with total possible subscore of 0 (best) to 6 (worst) and possible total score of 0 (best) to 18 (worst): 1) Clinical

Symptoms: Stool Frequency (0=usual to postoperative stool frequency to 2=3 or more stools/day>postoperative usual); Rectal bleeding (0=None or rare, 1=Present daily); Fecal urgency/abdominal cramps (0=None to 2=Usual), Fever [temperature>37.8 degrees C.] (0=Absent, 1=Present); 2) Endoscopic Inflammation: Edema, Granularity, Friability, Loss of vascular pattern, Mucous exudates, Ulcerations. Each item is scored on scale of 0=not present to 1=present summed up to an endoscopic subscore=0 (best) to 6 (worst); 3) Acute Histologic Inflammation: Polymorphic nuclear leukocyte infiltration (0=None to 3=Severe plus crypt abscess), Ulceration per low power field [mean] (0=0% to 3=>50%), higher scores=more severe disease.
4. Achieving a partial response. Percentage of subjects achieving a partial response (defined as a reduction of mPDAI score by at least 2 points from baseline) at weeks 14 and 34.

Partial mPDAI response was defined as a reduction in mPDAI score by ≥2 points from Baseline. The 12-point mPDAI score was calculated as a sum of from two 6-point subscales, where 0=best and 12=worst: 1) Clinical Symptoms: Stool Frequency (0=usual to postoperative stool frequency to 2=3 or more stools/day>postoperative usual); Rectal bleeding (0=None or rare to 1=Present daily); Fecal urgency or abdominal cramps (0=None to 2=Usual), Fever [temperature ≥37.8 degrees C.] (0=Absent and 1=Present) for a clinical symptoms subscore of 0 (best) to 6 (worst); 2) Endoscopic Inflammation Findings: Edema, Granularity, Friability, Loss of vascular pattern, Mucous exudates and Ulcerations. Each item was scored on a scale of 0=not present to 1=present summed up to an endoscopic subscore ranging from 0 (best) to 6 (worst) where higher scores indicated more severe disease. Last observation carried forward (LOCF) method was used for analyses.
5. Change From Baseline in PDAI Endoscopic Inflammation Subscore at Weeks 14 and 34

The PDAI Endoscopic Inflammation subscore is a sum of scores from findings for Edema, Granularity, Friability, Loss of vascular pattern, Mucous exudates, and Ulcerations, each scored on 0=not present to 1=present scale summed up to a subscore ranging from 0 (best) to 6 (worst) where higher scores=more severe disease. A negative change from Baseline indicated improvement. LOCF method was used for analyses.
6. Change From Baseline in PDAI Acute Histologic Inflammation Subscore at Weeks 14 and 34

The PDAI Acute Histologic Inflammation subscore was a sum score from findings for Polymorphic nuclear leukocyte infiltration (0=None to 3=Severe plus crypt abscess), and Ulceration per low power field [mean] (0-0% to 3=>50%) summed up to a subscore ranging from 0 (best) to 6 (worst) where higher scores=more severe disease. A negative change from Baseline indicated improvement. LOCF method was used for analyses.
7. Change From Baseline in Total PDAI Score at Weeks 14 and 34.

The 18-point PDAI score was calculated as a sum of three 6-point scales with total possible subscore of 0 (best) to 6 (worst) and possible total score of 0 (best) to 18 (worst): 1) Clinical Symptoms: Stool Frequency (0=usual to postoperative stool frequency to 2=3 or more stools/day>postoperative usual); Rectal bleeding (0=None or rare, 1=Present daily); Fecal urgency/abdominal cramps (0=None to 2=Usual), Fever [temperature>37.8 degrees C.] (0=Absent, 1=Present); 2) Endoscopic Inflammation: Edema, Granularity, Friability, Loss of vascular pattern, Mucous exudates, Ulcerations. Each item was scored on scale of 0=not present to 1=present summed up to an endoscopic subscore ranging from 0 (best) to 6 (worst); 3) Acute Histologic Inflammation: Polymorphic nuclear leukocyte infiltration (0=None to 3=Severe plus crypt abscess), Ulceration per low power field [mean] (0=0% to 3=>50%) where higher scores=more severe disease. A negative change from Baseline indicated improvement. LOCF method was used for analyses.
8. Change From Baseline in Inflammatory Bowel Disease Questionnaire (IBDQ) Total Score at Weeks 14, 22, and 34.

The IBDQ was an instrument used to assess quality of life in adult participants with inflammatory bowel disease (IBD). It included 32 questions on 4 domains of Health-Related Quality-of-Life (HRQOL): Bowel Systems (10 items), Emotional Function (12 items), Social Function (5 items), and Systemic Function (5 items). Participants were asked to recall symptoms and quality of life from the last 2 weeks and rate each item on a 7-point Likert scale (1=worst to 7=best). A total IBDQ score was calculated by summing the scores from each domain; the total IBDQ score ranged from 32 to 224, with lower scores reflecting worse HRQOL. A negative change from Baseline indicated worsening. LOCF method was used for analyses.
9. Change From Baseline in Cleveland Global Quality of Life (CGQL) at Weeks 14, 22, and 34.

The CGQL (Fazio score) was a quality-of-life indicator specifically for participants with ileal pouch-anal anastomosis. Participants rated 3 items (current quality of life, current quality of health, and current energy level), each on a scale of 0 to 10 (0=worst; 10=best). The scores were added, and the final CGQL utility score was obtained by dividing this result by 30. The total score ranged from 0 (worst) to 1 (best) where lower scores indicated less quality of life. A negative change from Baseline indicated worsening. LOCF method was used for analyses.

Table 2 provides baseline statistics. Participant flow is described in Table 3. Primary and secondary outcome results are provided in Table 4. Adverse events are provided in Table 5.

### Primary and Secondary Outcome Results

Among the 102 patients who underwent randomization, the incidence of mPDAI-defined remission at week 14 was 31.4% (16 of 51 patients) with vedolizumab versus 9.8% (5 of 51 patients) with placebo (difference, 21.6 percentage points; 95% confidence interval [CI], 4.9 to 37.5; P=0.013).

Vedolizumab also proved effective when patients were evaluated for mPDAI-defined remission at week 34. The incidence of mPDAI-defined remission at week 34 was 35.3% (18 of 51 patients) with vedolizumab versus 17.6% (9 of 51 patients) with placebo (difference, 17.6 percentage points; 95% CI, 0.3 to 35.1; P=0.043).

Patients receiving vedolizumab also showed PDAI-defined remission at week 14 and week 34. The incidence of PDAI-defined remission at week 14 was 35.3% (18 of 51 patients) with vedolizumab versus 9.8% (5 of 51 patients) with placebo (difference, 25.5 percentage points; 95% CI, 8.0 to 41.4; P=0.004). The incidence of PDAI-defined remission at week 34 was 37.3% (19 of 51 patients) with vedolizumab in comparison to 17.6% (9 of 51 patients) with placebo (difference, 19.6 percentage points; 95% CI, 1.9 to 37.0; P=0.027).

Median time from start of treatment to PDAI remission was 239 days in the vedolizumab group. Due to the low number of participants with events, the median time from start of treatment to PDAI remission was not estimated in the placebo group (hazard ratio for achieving PDAI remission: 3.95; 95% CI, 1.7-9.4).

At week 14, 62.7% of the patients in the vedolizumab group achieved a partial mPDAI response (difference, 29.4 percentage points; 95% CI, 8.0 to 47.6; P=0.003) in comparison to 33.3% of the patients in the placebo group. At week 34, 51% of the patients in the vedolizumab group compared to 29.4% of the patients in the placebo group achieved a partial mPDAI response (difference, 21.6 percentage points; 95% CI, 1.9 to 39.8; P=0.026).

With respect to change from baseline in PDAI endoscopic inflammation subscore, patients in the vedolizumab group had a −1.1 change in score from baseline at week 14 compared to a −0.2 change in score from baseline at week 14 in the placebo group (Odds estimator 2.02; 95% CI, 1.11 to 2.93; P=0.002). At week 34, the vedolizumab group had a −1.2 change in score from baseline compared to a −0.6 change in score from baseline in the placebo group (Odds estimator 1.71; 95% CI, 0.92 to 2.49; P=0.020).

With respect to change from baseline in PDAI acute histologic inflammation subscore, patients in the vedolizumab group had a −0.4 change in score from baseline at week 14 compared to a −0.1 change in score from baseline at week 14 in the placebo group (Odds estimator 1.34; 95% CI, 0.74 to 1.94; P=0.191). At week 34, the vedolizumab group had a −0.2 change in score from baseline, and the placebo group also had a −0.2 change in score from baseline (Odds estimator 1.07; 95% CI, 0.60 to 1.54; P=0.766).

For change from baseline in total PDAI score, patients in the vedolizumab group had a −2.7 change in score from baseline at week 14 compared to a −1.3 change in score from baseline at week 14 in the placebo group (Odds estimator 1.57; 95% CI, 0.83 to 2.31; P=0.055). At week 34, the vedolizumab group had a −2.9 change in score from baseline compared to a −1.6 change in score from baseline in the placebo group (Odds estimator 1.48; 95% CI, 0.79 to 2.16; P=0.095).

Change from baseline in Inflammatory Bowel Disease Questionnaire (IBDQ) Total Score at Weeks 14, 22, and 34 was assessed. At week 14, patients in the vedolizumab group had a 18.3 change in score from baseline and patients in the placebo group had a 14.6 change in score from baseline (Odds estimator 1.14; 95% CI, 0.61 to 1.67; P=0.575). At week 22, patients in the vedolizumab group had a 21.3 change in score from baseline and patients in the placebo group had a 16.0 change in score from baseline (Odds estimator 1.21; 95% CI, 0.65 to 1.78; P=0.403). At week 34, patients in the vedolizumab group had a 24.4 change in score from baseline and patients in the placebo group had a 10.4 change in score from baseline (Odds estimator 1.60; 95% CI, 0.85 to 2.34; P=0.047).

Change from baseline in Cleveland Global Quality of Life (CGQL) at Weeks 14, 22, and 34 was also assessed. At week 14, patients in the vedolizumab group had a 0.088 change in score from baseline and patients in the placebo group had a 0.051 change in score from baseline (Odds estimator 1.44; 95% CI, 0.77 to 2.12; P=0.119). At week 22, patients in the vedolizumab group had a 0.093 change in score from baseline and patients in the placebo group had a 0.073 change in score from baseline (Odds estimator 1.15; 95% CI, 0.62 to 1.69; P=0.542). At week 34, patients in the vedolizumab group had a 0.083 change in score from baseline and patients in the placebo group had a 0.056 change in score from baseline (Odds estimator 1.22; 95% CI, 0.65 to 1.78; P=0.404).

All-cause mortality was 0% in both the vedolizumab group and the placebo group. The time frame for observing serious adverse effects was from the first dose of the study drug up to 18 weeks after the last dose (up to 50 weeks). The investigator documented any occurrence of adverse events and abnormal laboratory findings at each visit. Any event spontaneously reported by the participant or observed by the investigator was recorded, irrespective of the relation to study treatment. Serious adverse events occurred in 3 of 51 patients (5.88%) in the vedolizumab group and in 4 of 51 patients (7.84%) in the placebo group. Other (not including serious) adverse events occurred in 42 of 51 patients (82.35%) in the vedolizumab group and in 35 of 51 patients (68.63%) in the placebo group.

In this randomized placebo-controlled trial, vedolizumab demonstrated efficacy and safety/tolerability over 34 weeks in patients with an IPAA (ileal-pouch anal anastomosis) for UC and chronic pouchitis.

TABLE 2
Baseline Characteristics

| Arm/Group Title | | Placebo IV | | Vedolizumab IV 300 mg | | Total | |
|---|---|---|---|---|---|---|---|
| ▾ Arm/Group Description | | Vedolizumab placebo- | | Vedolizumab 300 mg, IV | | Total of all | |
| | | matching IV infusion, once | | infusion, once at Day 1, | | reporting groups | |
| | | at Day 1, Weeks 2, 6, 14, | | Weeks 2, 6, 14, 22, and 30 | | | |
| | | 22, and 30 along with | | along with ciprofloxacin | | | |
| | | ciprofloxacin 500 mg, | | 500 mg, tablet, orally | | | |
| | | tablet, orally twice daily up | | twice daily up to Week 4. | | | |
| | | to Week 4. | | | | | |
| Overall Number of Baseline | | 51 | | 51 | | 102 | |
| Participants | | | | | | | |
| ▾ Baseline Analysis Population | | Full Analysis Set included all randomized participants who received at least 1 dose of study | | | | | |
| Description | | medication, as randomized. | | | | | |
| Age, Continuous | | | | | | | |
| Mean (Standard | | | | | | | |
| Deviation) | | | | | | | |
| Unit of | | | | | | | |
| measure: Years | | | | | | | |
| | Number | 51 participants | | 51 participants | | 102 participants | |
| | Analyzed | | | | | | |
| | | 42.9 | (13.48) | 40.8 | (11.32) | 41.9 | (12.44) |
| Sex: Female, Male | | | | | | | |
| Measure Type: Count | | | | | | | |
| of Participants | | | | | | | |
| Unit of | | | | | | | |
| measure: Participants | | | | | | | |
| | Number | 51 participants | | 51 participants | | 102 participants | |
| | Analyzed | | | | | | |
| | Female | 13 | (25.5%) | 19 | (37.3%) | 32 | (31.4%) |
| | Male | 38 | (74.5%) | 32 | (62.7%) | 70 | (68.6%) |
| Region of | Number | 51 participants | | 51 participants | | 102 participants | |
| Enrollment | Analyzed | | | | | | |
| Measure Type: Count | | | | | | | |
| of Participants | | | | | | | |
| Unit of | | | | | | | |
| measure: Participants | | | | | | | |
| Canada | | 7 | (13.7%) | 9 | (17.6%) | 16 | (15.7%) |
| United States | | 11 | (21.6%) | 12 | (23.5%) | 23 | (22.5%) |
| Belgium | | 6 | (11.8%) | 1 | (2.0%) | 7 | (6.9%) |
| France | | 2 | (3.9%) | 5 | (9.8%) | 7 | (6.9%) |
| Germany | | 1 | (2.0%) | 2 | (3.9%) | 3 | (2.9%) |
| Italy | | 10 | (19.6%) | 10 | (19.6%) | 20 | (19.6%) |
| Netherlands | | 6 | (11.8%) | 4 | (7.8%) | 10 | (9.8%) |
| Spain | | 1 | (2.0%) | 0 | (0.0%) | 1 | (1.0%) |
| United Kingdom | | 7 | (13.7%) | 8 | (15.7%) | 15 | (14.7%) |

| | | | | |
|---|---|---|---|---|
| Height | | | | |
| Mean (Standard | | | | |
| Deviation) | | | | |
| Unit of measure: Cm | | | | |
| | Number | 51 participants | 51 participants | 102 participants |
| | Analyzed | | | |

| | | | | | |
|---|---|---|---|---|---|
| 175.3 | (9.10) | 172.4 | (10.79) | 173.8 | (10.04) |

| | | | | |
|---|---|---|---|---|
| Weight | | | | |
| Mean (Standard | | | | |
| Deviation) | | | | |
| Unit of measure: Kg | | | | |
| Body Mass Index | Number | 51 participants | 51 Participants | 102 Participants |
| (BMI) ^{[1]} | Analyzed | | | |
| Mean (Standard | | 79.60 (19.115) | 72.13 (17.588) | 75.87 (18.658) |
| Deviation) | | | | |
| Unit of | | | | |
| Measure: Kg/m^{Λ}2 | | | | |
| | Number | 51 participants | 51 Participants | 102 Participants |
| | Analyzed | 25.74 (5.125) | 24.13 (4.891) | 24.93 (5.049) |

^{[1]} Measure Description: BMI = weight (kg)/(m)^{Λ}2

TABLE 3
Participant Flow

| | |
|---|---|
| Pre-assignment Details | Participants with a diagnosis of chronic or recurrent pouchitis were enrolled in a 1:1 ratio to receive placebo IV or |
| | vedolizumab IV 300 mg. |

| Arm/Group Title | Placebo IV | Vedolizumab IV 300 mg |
|---|---|---|
| Arm/Group Description | Vedolizumab placebo-matching IV infusion, once at | Vedolizumab 300 mg, IV infusion, once at Day 1, |
| | Day 1, Weeks 2, 6, 14, 22, and 30 along with | Weeks 2, 6, 14, 22, and 30 along with ciprofloxacin |
| | ciprofloxacin 500 mg, tablet, orally twice daily up to | 500 mg, tablet, orally twice daily up to Week 4. |
| | Week 4. | |

Period Title: Overall Study

| | | |
|---|---|---|
| Started | 51 | 51 |
| Completed | 30 | 32 |
| Not Completed | 21 | 19 |
| Reason Not Completed | | |
| Adverse Event | 5 | 2 |
| Significant Protocol Deviation | 0 | 1 |
| Lost to Follow-up | 1 | 0 |
| Voluntary Withdrawal | 8 | 9 |
| Lack of Efficacy | 6 | 7 |
| Reason not Specified | 1 | 0 |

TABLE 4
Outcome Measures: Primary and Secondary
1. Primary Outcome

| | |
|---|---|
| Title | Percentage of Participants With Chronic or Recurrent Pouchitis Achieving Clinically Relevant Remission at Week 14 |
| ▾ Description | Clinically relevant remission was defined as modified Pouchitis Disease Activity Index (mPDAI) score <5 and a |
| | reduction of mPDAI score by ≥2 points from Baseline. The 12-point mPDAI score was calculated as a sum of from two |
| | 6-point subscales, where 0 = best and 12 = worst.: 1) Clinical Symptoms: Stool Frequency (0 = usual to postoperative |
| | stool frequency to 2 = three or more stools/day > postoperative usual); Rectal bleeding (0 = None or rare to 1 = Present daily); |
| | Fecal urgency or abdominal cramps (0 = None to 2 = Usual), Fever [temperature >37.8 degrees C.] (0 = Absent and |
| | 1 = Present) for a clinical symptoms subscore of 0 (best) to 6 (worst); 2) Endoscopic Inflammation Findings: Edema, |
| | Granularity, Friability, Loss of vascular pattern, Mucous exudates and Ulcerations. Each item was scored on a scale of |
| | 0 = not present to 1 = present summed up to an endoscopic subscore ranging from 0 (best) to 6 (worst) where higher scores |
| | indicated more severe disease. |
| Time Frame | Week 14 |

| ▾ Outcome Measure Data | | |
|---|---|---|
| ▾ Analysis Population Description | | |
| Full Analysis Set (FAS) included all randomized participants who received at least 1 dose of study medication, as randomized. | | |
| Arm/Group Title | Placebo IV | Vedolizumab IV 300 mg |
| ▾ Arm/Group | Vedolizumab placebo-matching IV infusion, once at Day 1, Weeks | Vedolizumab 300 mg, IV infusion, once at Day 1, Weeks 2, 6, |
| Description: | 2, 6, 14, 22, and 30 along with ciprofloxacin 500 mg, tablet, orally | 14, 22, and 30 along with ciprofloxacin 500 mg, tablet, orally |
| | twice daily up to Week 4. | twice daily up to Week 4. |
| Overall Number of | 51 | 51 |
| Participants Analyzed | | |
| Measure Type: Number | | |
| Number (95% | | |
| Confidence Interval) | | |
| Unit of Measure: | | |
| percentage of | | |
| participants | | |
| | 9.8 | 31.4 |
| | (3.3 to 21.4) | (19.1 to 45.9) |

▾ Statistical Analysis 1

| | | |
|---|---|---|
| Statistical Analysis | Comparison Group | Placebo IV, Vedolizumab IV 300 mg |
| Overview | Selection | |
| | Comments | The Placebo IV and Vedolizumab IV 300 mg groups were analyzed using Fisher's Exact Test at Week |
| | | 14. |
| | Type of Statistical Test | Superiority |
| Statistical Test of | P-Value | 0.013 |
| Hypothesis | Comments | The significance level was 0.05. |
| | Method | Fisher's Exact Test |
| | Comments | [Not Specified] |
| Method of Estimation | Estimation Parameter | Percentage Difference |
| | Estimated Value | 21.6 |
| | Confidence Interval | (2-Sided) 95% |
| | | 4.9 to 37.5 |

2. Secondary Outcome

| | |
|---|---|
| Title | Percentage of Participants With Chronic or Recurrent Pouchitis Achieving Clinically Relevant Remission at |
| | Week 34 |
| ▾ Description | Clinically relevant remission was defined as mPDAI score <5 and a reduction of mPDAI score by ≥2 points |
| | from Baseline. The 12-point mPDAI score was calculated as a sum of from two 6-point subscales, where 0 = |
| | best and 12 = worst.: 1) Clinical Symptoms: Stool Frequency (0 = usual to postoperative stool frequency to |
| | 2 = 3 or more stools/day > postoperative usual); Rectal bleeding (0 = None or rare to 1 = Present daily); Fecal |
| | urgency or abdominal cramps (0 = None to 2 = Usual), Fever [temperature >37.8 degrees C.] (0 = Absent and |
| | 1 = Present) for a clinical symptoms subscore of 0 (best) to 6 (worst); 2) Endoscopic Inflammation Findings: |
| | Edema, Granularity, Friability, Loss of vascular pattern, Mucous exudates and Ulcerations. Each item was |
| | scored on a scale of 0 = not present to 1 = present summed up to an endoscopic subscore ranging from 0 (best) |
| | to 6 (worst) where higher scores indicated more severe disease. |
| Time Frame | Week 34 |

| ▾ Outcome Measure Data | | |
|---|---|---|
| ▾ Analysis Population Description | | |
| FAS included all randomized participants who received at least 1 dose of study medication, as randomized. | | |
| Arm/Group Title | Placebo IV | Vedolizumab IV 300 mg |
| ▾ Arm/Group | Vedolizumab placebo-matching intravenous (IV) infusion, once at | Vedolizumab 300 mg, IV infusion, once at Day 1, Weeks 2, |
| Description: | Day 1, Weeks 2, 6, 14, 22, and 30 along with ciprofloxacin 500 mg, | 6, 14, 22, and 30 along with ciprofloxacin 500 mg, tablet, |
| | tablet, orally twice daily up to Week 4. | orally twice daily up to Week 4. |
| Overall Number of | 51 | 51 |
| Participants Analyzed | | |
| Measure Type: Number | | |
| Number (95% | | |
| Confidence Interval) | | |
| Unit of Measure: | | |
| percentage of | | |
| participants | | |
| | 17.6 | 35.3 |
| | (8.4 to 30.9) | (22.4 to 49.9) |

| ▾ Statistical Analysis 1 | | |
|---|---|---|
| Statistical Analysis | Comparison Group | Placebo IV, Vedolizumab IV 300 mg |
| Overview | Selection | |
| | Type of Statistical Test | Superiority |
| Statistical Test of | P-Value | =0.043 |
| Hypothesis | Comments | No multiplicity adjustment for inferential testing for secondary endpoints was preplanned, p-values from these |
| | | tests are presented as nominal p-values. The significance level was 0.05. |
| | Method | Chi-squared Test |
| Method of | Estimation Parameter | Percentage Difference |
| Estimation | Estimated Value | 17.6 |
| | Confidence Interval | (2-Sided) 95% |
| | | 0.3 to 35.1 |

3. Secondary Outcome

| | |
|---|---|
| Title | Percentage of Participants Achieving Pouchitis Disease Activity Index (PDAI) Remission at Weeks 14 and 34 |
| ▾ Description | PDAI remission was PDAI score <7 and a reduction of PDAI score by ≥3 points from Baseline. The 18-point PDAI |
| | score was calculated as a sum of three 6-point scales with total possible subscore of 0 (best) to 6 (worst) and possible |
| | total score of 0 (best) to 18 (worst): 1) Clinical Symptoms: Stool Frequency (0 = usual to postoperative stool frequency to |
| | 2 = 3 or more stools/day > postoperative usual); Rectal bleeding (0 = None or rare, 1 = Present daily); Fecal |
| | urgency/abdominal cramps (0 = None to 2 = Usual), Fever [temperature >37.8 degrees C.] (0 = Absent, 1 = Present); 2) |
| | Endoscopic Inflammation: Edema, Granularity, Friability, Loss of vascular pattern, Mucous exudates, Ulcerations. Each |
| | item was scored on scale of 0 = not present to 1 = present summed up to an endoscopic subscore ranging from 0 (best) to 6 |
| | (worst); 3) Acute Histologic Inflammation: Polymorphic nuclear leukocyte infiltration (0 = None to 3 = Severe plus crypt |
| | abscess), Ulceration per low power field [mean] (0 = 0% to 3 => 50%) where higher scores indicated more severe disease. |
| Time Frame | Weeks 14 and 34 |

| ▾ Outcome Measure Date | | |
|---|---|---|
| ▾ Analysis Population Description | | |
| FAS included all randomized participants who received at least 1 dose of study medication, as randomized. | | |
| Arm/Group Title | Placebo IV | Vedolizumab IV 300 mg |
| ▾ Arm/Group | Vedolizumab placebo-matching intravenous (IV) infusion, once at | Vedolizumab 300 mg, IV infusion, once at Day 1, Weeks 2, |
| Description: | Day 1, Weeks 2, 6, 14, 22, and 30 along with ciprofloxacin 500 mg, | 6, 14, 22, and 30 along with ciprofloxacin 500 mg, tablet, |
| | tablet, orally twice daily up to Week 4. | orally twice daily up to Week 4. |
| Overall Number of | 51 | 51 |
| Participants Analyzed | | |
| Measure Type: Number | | |
| Number (95% | | |
| Confidence Interval) | | |
| Unit of Measure: | | |
| percentage of | | |
| participants | | |
| Week 14 | 9.8 | 35.3 |
| | (3.3 to 21.4) | (22.4 to 49.9) |
| Week 34 | 17.6 | 37.3 |
| | (8.4 to 30.9) | (24.1 to 51.9) |

| ▾ Statistical Analysis 1 | | |
|---|---|---|
| Statistical Analysis | Comparison Group | Placebo IV, Vedolizumab IV 300 mg |
| Overview | Selection | |
| | Comments | Week 14 |
| | Type of Statistical Test | Superiority |
| | Comments | [Not Specified] |
| Statistical Test of | P-Value | =0.004 |
| Hypothesis | Comments | No multiplicity adjustment for inferential testing for secondary endpoints was preplanned, p-values from these |
| | | tests are presented as nominal p-values. The significance level was 0.05. |
| | Method | Fisher's Exact Test |
| Method of | Estimation Parameter | Percentage Difference |
| Estimation | Estimated Value | 25.5 |
| | Confidence Interval | (2-Sided) 95% |
| | | 8.0 to 41.4 |
| ▾ Statistical Analysis 2 | | |
| Statistical Analysis | Comparison Group | Placebo IV, Vedolizumab IV 300 mg |
| Overview | Selection | |
| | Comments | Week 34. |
| | Type of Statistical Test | Superiority |
| Statistical Test of | P-Value | =0.027 |
| Hypothesis | Comments | No multiplicity adjustment for inferential testing for secondary endpoints was preplanned, p-values from these |
| | | tests are presented as nominal p-values. The significance level was 0.05. |
| | Method | Chi-squared Test |
| Method of | Estimation Parameter | Percentage Difference |
| Estimation | Estimated Value | 19.6 |
| | Confidence Interval | (2-Sided) 95% |
| | | 1.9 to 37.0 |

4. Secondary Outcome

| | |
|---|---|
| Title | Time to PDAI Remission |
| ▾ Description | Time to remission-time in days from start of treatment to PDAI Remission (PDAI score <7 and decrease in PDAI score ≥3 |
| | points from Baseline). 18-point PDAI score-calculated as sum of 3, 6-point scales with total possible subscore of 0 |
| | (best) to 6 (worst) and possible total score of 0 (best) to 18 (worst): 1) Clinical Symptoms: Stool Frequency(0 = usual to |
| | postoperative stool frequency to 2 = 3 or more stools/day > postoperative usual); Rectal bleeding (0 = None or |
| | rare, 1 = Present daily); Fecal urgency/abdominal cramps (0 = None to 2 = Usual), Fever [temperature >37.8 degrees C.] |
| | (0 = Absent, 1 = Present); 2) Endoscopic Inflammation: Edema, Granularity, Friability, Loss of vascular pattern, Mucous |
| | exudates, Ulcerations. Each item was scored on scale of 0 = not present to 1 = present summed up to an endoscopic |
| | subscore = 0 (best) to 6 (worst); 3) Acute Histologic Inflammation: Polymorphic nuclear leukocyte infiltration (0 = None to |
| | 3 = Severe plus crypt abscess), Ulceration per low power field [mean] (0 = 0% to 3 => 50%), higher scores = more severe |
| | disease. |
| Time Frame | Baseline up to Week 34 |

| ▾ Outcome Measure Data | | |
|---|---|---|
| ▾ Analysis Population Description | | |
| FAS included all randomized participants who received at least 1 dose of study medication, as randomized. | | |
| Arm/Group Title | Placebo IV | Vedolizumab IV 300 mg |
| ▾ Arm/Group | Vedolizumab placebo-matching intravenous (IV) infusion, once at | Vedolizumab 300 mg, IV infusion, once at Day 1, Weeks 2, 6, |
| Description: | Day 1, Weeks 2, 6, 14, 22, and 30 along with ciprofloxacin 500 mg, | 14, 22, and 30 along with ciprofloxacin 500 mg, tablet, orally |
| | tablet, orally twice daily up to Week 4. | twice daily up to Week 4. |
| Overall Number of | 51 | 50 |
| Participants Analyzed | | |
| Median (95% | | |
| Confidence Interval) | | |
| Unit of Measure: days | | |
| | NA ^{[1]} | 239.0 ^{[2]} |
| | (NA to NA) | (101.0 to NA) |

^{[1]} Median and 95% Confidence Interval (CI) was not estimated due to the low number of participants with events.
^{[2]} Upper limit of 95% CI was not estimated due do the low number of participants with events.

▾ Statistical Analysis 1

| | | |
|---|---|---|
| Statistical Analysis | Comparison Group | Placebo IV, Vedolizumab IV 300 mg |
| Overview | Selection | |
| | Type of Statistical Test | Superiority |
| | Comments | [Not Specified] |
| Method of | Estimation Parameter | Hazard Ratio (HR) |
| Estimation | Estimated Value | 3.95 |
| | Confidence Interval | (2-Sided) 95% |
| | | 1.7 to 9.4 |
| | Estimation Comments | Hazard ratio for achieving PDAI remission. |

5. Secondary Outcome

| Title | | Percentage of Participants Achieving a Partial mPDAI Response at Weeks 14 and 34 | |
|---|---|---|---|
| ▾ Description | | Partial mPDAI response was defined as a reduction in mPDAI score by ≥2 points from Baseline. The 12-point mPDAI | |
| | | score was calculated as a sum of from two 6-point subscales, where 0 = best and 12 = worst: 1) Clinical Symptoms: | |
| | | Stool Frequency (0 = usual to postoperative stool frequency to 2 = 3 or more stools/day > postoperative usual); Rectal | |
| | | bleeding (0 = None or rare to 1 = Present daily); Fecal urgency or abdominal cramps (0 = None to 2 = Usual), Fever | |
| | | [temperature >37.8 degrees C.] (0 = Absent and 1 = Present) for a clinical symptoms subscore of 0 (best) to 6 (worst); 2) | |
| | | Endoscopic Inflammation Findings: Edema, Granularity, Friability, Loss of vascular pattern, Mucous exudates and | |
| | | Ulcerations. Each item was scored on a scale of 0 = not present to 1 = present summed up to an endoscopic subscore | |
| | | ranging from 0 (best) to 6 (worst) where higher scores indicated more severe disease. Last observation carried forward | |
| | | (LOCF) method was used for analyses. | |
| Time Frame | | Weeks 14 and 34 | |
| | ▾ Outcome Measure Data | | |
| | ▾ Analysis Population Description | | |
| | FAS included all randomized participants who received at least 1 dose of study medication, and randomized. | | |
| | Arm/Group Title | Placebo IV | Vedolizumab IV 300 mg |
| | ▾ Arm/Group | Vedolizumab placebo-matching intravenous (IV) infusion, once at | Vedolizumab 300 mg, IV infusion, once at Day 1, Weeks 2, |
| | Description: | Day 1, Weeks 2, 6, 14, 22, and 30 along with ciprofloxacin 500 mg, | 6, 14, 22, and 30 along with ciprofloxacin 500 mg, tablet, |
| | | tablet, orally twice daily up to Week 4. | orally twice daily up to Week 4. |
| | Overall Number of | 51 | 51 |
| | Participants Analyzed | | |
| | Measure Type: Number | | |
| | Number (95% | | |
| | Confidence Interval) | | |
| | Unit of Measure: | | |
| | percentage of | | |
| | participants | | |
| | Week 14 | 33.3 | 62.7 |
| | | (20.8 to 47.9) | (48.1 to 75.9) |
| | Week 34 | 29.4 | 51.0 |
| | | (17.5 to 43.8) | (36.6 to 65.2) |

| ▾ Statistical Analysis 1 | | |
|---|---|---|
| Statistical Analysis | Comparison Group | Placebo IV, Vedolizumab IV 300 mg |
| Overview | Selection | |
| | Comments | Week 14 |
| | Type of Statistical Test | Superiority |
| Statistical Test of | P-Value | =0.003 |
| Hypothesis | Comments | No multiplicity adjustment for inferential testing for secondary endpoints was preplanned, p-values from these |
| | | tests are presented as nominal p-values. The significance level was 0.05. |
| | Method | Chi-squared Test |
| Method of | Estimation Parameter | Percentage Difference |
| Estimation | Estimated Value | 29.4 |
| | Confidence Interval | (2-Sided) 95% |
| | | 8.0 to 47.6 |
| ▾ Statistical Analysis 2 | | |
| Statistical Analysis | Comparison Group | Placebo IV, Vedolizumab IV 300 mg |
| Overview | Selection | |
| | Comments | Week 34 |
| | Type of Statistical Test | Superiority |
| Statistical Test of | P-Value | =0.026 |
| Hypothesis | Comments | No multiplicity adjustment for inferential testing for secondary endpoints was preplanned, p-values from these |
| | | tests are presented as nominal p-values. The significance level was 0.05. |
| | Method | Chi-squared Test |
| | Comments | [Not Specified] |
| Method of | Estimation Parameter | Percentage Difference |
| Estimation | Estimated Value | 21.6 |
| | Confidence Interval | (2-Sided) 95% |
| | | 1.9 to 39.8 |

6. Secondary Outcome

| | |
|---|---|
| Title | Change From Baseline in PDAI Endoscopic Inflammation Subscore at Weeks 14 and 34 |
| ▾ Description | The PDAI Endoscopic Inflammation subscore was a sum of scores from findings for Edema, Granularity, Friability, |
| | Loss of vascular pattern, Mucous exudates, and Ulcerations, each scored on 0 = not present to 1 = present scale summed up |
| | to a subscore ranging from 0 (best) to 6 (worst) where higher scores = more severe disease. A negative change from |
| | Baseline indicated improvement. LOCF method was used for analyses. |
| Time Frame | Baseline up to Weeks 14 and 34 |

| ▾ Outcome Measure Data | | |
|---|---|---|
| ▾ Analysis Population Description | | |
| FAS included all randomized participants who received at least 1 dose of study medication, as randomized. | | |
| Arm/Group Title | Placebo IV | Vedolizumab IV 300 mg |
| ▾ Arm/Group | Vedolizumab placebo-matching intravenous (IV) infusion, once at | Vedolizumab 300 mg, IV infusion, once at Day 1, Weeks 2, 6, |
| Description: | Day 1, Weeks 2, 6, 14, 22, and 30 along with ciprofloxacin 500 mg, | 14, 22, and 30 along with ciprofloxacin 500 mg, tablet, orally |
| | tablet, orally twice daily up to Week 4. | twice daily up to Week 4. |
| Overall Number of | 51 | 51 |
| Participants Analyzed | | |
| Mean (Standard | | |
| Deviation) | | |
| Unit of Measure: score | | |
| on a scale | | |
| Baseline | 4.5 (1.36) | 4.6 (1.15) |
| Change from Baseline | −0.2 (1.36) | −1.1 (1.59) |
| at Week 14 | | |
| Change from Baseline | −0.6 (1.86) | −1.2 (1.87) |
| at Week 34 | | |

| ▾ Statistical Analysis 1 | | |
|---|---|---|
| Statistical Analysis | Comparison Group | Placebo IV, Vedolizumab IV 300 mg |
| Overview | Selection | |
| | Comments | Change from Baseline at Week 14 |
| | Type of Statistical Test | Superiority |
| Statistical Test of | P-Value | =0.002 |
| Hypothesis | Comments | No multiplicity adjustment for inferential testing for secondary endpoints was preplanned, p-values from these |
| | | tests are presented as nominal p-values. The significance level was 0.05. |
| | Method | Wilcoxon (Mann-Whitney) |
| | Comments | P-value is from Wilcoxon Rank-sum Test comparing change from Baseline to relevant visits. Wilcoxon-Mann- |
| | | Whitney odds estimator, 95% CI are presented. |
| Method of | Estimation Parameter | Odds Estimator |
| Estimation | Estimated Value | 2.02 |
| | Confidence Interval | (2-Sided) 95% |
| | | 1.11 to 2.93 |
| ▾ Statistical Analysis 2 | | |
| Statistical Analysis | Comparison Group | Placebo IV, Vedolizumab IV 300 mg |
| Overview | Selection | |
| | Comments | Change from Baseline at Week 34 |
| | Type of Statistical Test | Superiority |
| Statistical Test of | P-Value | =0.020 |
| Hypothesis | Comments | No multiplicity adjustment for inferential testing for secondary endpoints was preplanned, p-values from these |
| | | tests are presented as nominal p-values. The significance level was 0.05. |
| | Method | Wilcoxon (Mann-Whitney) |
| | Comments | P-value is from Wilcoxon Rank-sum Test comparing change from Baseline to relevant visits. Wilcoxon-Mann- |
| | | Whitney odds estimator, 95% CI are presented. |
| Method of | Estimation Parameter | Odds Estimator |
| Estimation | Estimated Value | 1.71 |
| | Confidence Interval | (2-Sided) 95% |
| | | 0.92 to 2.49 |

7. Secondary Outcome

| | |
|---|---|
| Title | Change From Baseline in PDAI Acute Histologic Inflammation Subscore at Weeks 14 and 34 |
| Description | The PDAI Acute Histologic Inflammation subscore was a sum score from findings for Polymorphic nuclear leukocyte |
| | infiltration (0 = None to 3 = Severe plus crypt abscess), and Ulceration per low power field [mean] (0 = 0% to 3 => 50%) |
| | summed up to a subscore ranging from 0 (best) to 6 (worst) where higher scores = more severe disease. A negative |
| | change from Baseline indicated improvement. LOCF method was used for analyses. |
| Time Frame | Baseline up to Weeks 14 and 34 |

| ▾ Outcome Measure Data | | |
|---|---|---|
| ▾ Analysis Population Description | | |
| FAS included all randomized participants who received at least 1 dose of study medication, as randomized. Overall number analyzed are the number of | | |
| participants with data evaluable for analyses. | | |
| Arm/Group Title | Placebo IV | Vedolizumab IV 300 mg |
| ▾ Arm/Group | Vedolizumab placebo-matching intravenous (IV) infusion, once at | Vedolizumab 300 mg, IV infusion, once at Day 1, Weeks 2, 6, |
| Description: | Day 1, Weeks 2, 6, 14, 22, and 30 along with ciprofloxacin 500 mg, | 14, 22, and 30 along with ciprofloxacin 500 mg, tablet, orally |
| | tablet, orally twice daily up to Week 4. | twice daily up to Week 4. |
| Overall Number of | 51 | 50 |
| Participants Analyzed | | |
| Mean (Standard | | |
| Deviation) | | |
| Unit of Measure: score | | |
| on a scale | | |
| Baseline | 2.6 (1.39) | 2.5 (1.42) |
| Change from Baseline | −0.1 (1.33) | −0.4 (1.98) |
| at Week 14 | | |
| Change from Baseline | −0.2 (1.52) | −0.2 (2.03) |
| at Week 34 | | |

| ▾ Statistical Analysis 1 | | |
|---|---|---|
| Statistical Analysis | Comparison Group | Placebo IV, Vedolizumab IV 300 mg |
| Overview | Selection | |
| | Comments | Change from Baseline at Week 14 |
| | Type of Statistical Test | Superiority |
| Statistical Test of | P-Value | =0.191 |
| Hypothesis | Comments | No multiplicity adjustment for inferential testing for secondary endpoints was preplanned, p-values from these |
| | | tests are presented as nominal p-values. The significance level was 0.05. |
| | Method | Wilcoxon (Mann-Whitney) |
| | Comments | P-value is from Wilcoxon Rank-sum Test comparing change from Baseline to relevant visits. Wilcoxon-Mann- |
| | | Whitney odds estimator, 95% CI are presented. |
| Method of | Estimation Parameter | Odds Estimator |
| Estimation | Estimated Value | 1.34 |
| | Confidence Interval | (2-Sided) 95% |
| | | 0.74 to 1.94 |
| ▾ Statistical Analysis 2 | | |
| Statistical Analysis | Comparison Group | Placebo IV, Vedolizumab IV 300 mg |
| Overview | Selection | |
| | Comments | Change from Baseline at Week 34 |
| | Type of Statistical Test | Superiority |
| Statistical Test of | P-Value | =0.766 |
| Hypothesis | Comments | No multiplicity adjustment for inferential testing for secondary endpoints was preplanned, p-values from these |
| | | tests are presented as nominal p-values. The significance level was 0.05. |
| | Method | Wilcoxon (Mann-Whitney) |
| | Comments | P-value is from Wilcoxon Rank-sum Test comparing change from Baseline to relevant visits. Wilcoxon-Mann- |
| | | Whitney odds estimator, 95% CI are presented. |
| Method of | Estimation Parameter | Odds Estimator |
| Estimation | Estimated Value | 1.07 |
| | Confidence Interval | (2-Sided) 95% |
| | | 0.60 to 1.54 |

8. Secondary Outcome

| | |
|---|---|
| Title | Change From Baseline in Total PDAI Score at Weeks 14 and 34 |
| ▾ Description | The 18-point PDAI score was calculated as a sum of three 6-point scales with total possible subscore of 0 (best) to 6 |
| | (worst) and possible total score of 0 (best) to 18 (worst): 1) Clinical Symptoms: Stool Frequency (0 = usual to |
| | postoperative stool frequency to 2 = 3 or more stools/day > postoperative usual); Rectal bleeding (0 = None or |
| | rare, 1 = Present daily); Fecal urgency/abdominal cramps (0 = None to 2 = Usual), Fever [temperature >37.8 degrees C.] |
| | (0 = Absent, 1 = Present); 2) Endoscopic Inflammation: Edema, Granularity, Friability, Loss of vascular pattern, Mucous |
| | exudates, Ulcerations. Each item was scored on scale of 0 = not present to 1 = present summed up to an endoscopic |
| | subscore ranging from 0 (best) to 6 (worst); 3) Acute Histologic Inflammation: Polymorphic nuclear leukocyte |
| | infiltration (0 = None to 3 = Severe plus crypt abscess), Ulceration per low power field [mean] (0 = 0% to 3 => 50%) where |
| | higher scores = more severe disease. A negative change from Baseline indicated improvement. LOCF method was used |
| | for analyses. |
| Time Frame | Baseline up to Weeks 14 and 34 |

| ▾ Outcome Measure Data | | |
|---|---|---|
| ▾ Analysis Population Description | | |
| FAS included all randomized participants who received at least 1 dose of study medication, as randomized. Overall number analyzed are the number of | | |
| participants with data for analyses. | | |
| Arm/Group Title | Placebo IV | Vedolizumab IV 300 mg |
| ▾ Arm/Group | Vedolizumab placebo-matching intravenous (IV) infusion, once at | Vedolizumab 300 mg, IV infusion, once at Day 1, Weeks 2, 6, |
| Description: | Day 1, Weeks 2, 6, 14, 22, and 30 along with ciprofloxacin 500 mg, | 14, 22, and 30 along with ciprofloxacin 500 mg, tablet, orally |
| | tablet, orally twice daily up to Week 4. | twice daily up to Week 4. |
| Overall Number of | 51 | 50 |
| Participants Analyzed | | |
| Mean (Standard | | |
| Deviation) | | |
| Unit of Measure: score | | |
| on a scale | | |
| Baseline | 10.5 (2.48) | 10.5 (2.20) |
| Change from Baseline | −1.3 (2.68) | −2.7 (3.86) |
| at Week 14 | | |
| Change from Baseline | −1.6 (3.41) | −2.9 (3.93) |
| at Week 34 | | |

| ▾ Statistical Analysis 1 | | |
|---|---|---|
| Statistical Analysis | Comparison Group | Placebo IV, Vedolizumab IV 300 mg |
| Overview | Selection | |
| | Type of Statistical Test | Superiority |
| Statistical Test of | P-Value | =0.055 |
| Hypothesis | Comments | No multiplicity adjustment for inferential testing for secondary endpoints was preplanned, p-values from these |
| | | tests are presented as nominal p-values. The significance level was 0.05. |
| | Method | Wilcoxon (Mann-Whitney) |
| | Comments | P-value is from Wilcoxon Rank-sum Test comparing change from Baseline to relevant visits. Wilcoxon-Mann- |
| | | Whitney odds estimator, 95% CI are presented. |
| Method of | Estimation Parameter | Odds Estimator |
| Estimation | Estimated Value | 1.57 |
| | Confidence Interval | (2-Sided) 95% |
| | | 0.83 to 2.31 |
| ▾ Statistical Analysis 2 | | |
| Statistical Analysis | Comparison Group | Placebo IV, Vedolizumab IV 300 mg |
| | Selection | |
| Overview | Comments | Change from Baseline at Week 34 |
| | Type of Statistical Test | Superiority |
| Statistical Test of | P-Value | =0.095 |
| Hypothesis | Comments | No multiplicity adjustment for inferential testing for secondary endpoints was preplanned, p-values from these |
| | | tests are presented as nominal p-values. The significance level was 0.05. |
| | Method | Wilcoxon (Mann-Whitney) |
| | Comments | P-value is from Wilcoxon Rank-sum Test comparing change from Baseline to relevant visits. Wilcoxon-Mann- |
| | | Whitney odds estimator, 95% CI are presented. |
| Method of | Estimation Parameter | Odds Estimator |
| Estimation | Estimated Value | 1.48 |
| | Confidence Interval | (2-Sided) 95% |
| | | 0.79 to 2.16 |

9. Secondary Outcome

| | |
|---|---|
| Title | Change From Baseline in Inflammatory Bowel Disease Questionnaire (IBDQ) Total Score at Weeks 14, 22, and 34 |
| ▾ Description | The IBDQ was an instrument used to assess quality of life in adult participants with inflammatory bowel disease (IBD). |
| | It included 32 questions on 4 domains of Health-Related Quality-of-Life (HRQOL): Bowel Systems (10 items), |
| | Emotional Function (12 items), Social Function (5 items), and Systemic Function (5 items). Participants were asked to |
| | recall symptoms and quality of life from the last 2 weeks and rate each item on a 7-point Likert scale (1 = worst to |
| | 7 = best). A total IBDQ score was calculated by summing the scores from each domain; the total IBDQ score ranges from |
| | 32 to 224, with lower scores reflecting worse HRQOL. A negative change from Baseline indicated worsening. LOCF |
| | method was used for analyses. |
| Time Frame | Baseline up to Weeks 14, 22, and 34 |

| ▾ Outcome Measure Data | | |
|---|---|---|
| ▾ Analysis Population Description | | |
| FAS included all randomized participants who received at least 1 dose of study medication, as randomized. Overall number analyzed are the number of | | |
| participants with data evaluable for analyses. | | |
| Arm/Group Title | Placebo IV | Vedolizumab IV 300 mg |
| ▾ Arm/Group | Vedolizumab placebo-matching intravenous (IV) infusion, once at | Vedolizumab 300 mg, IV infusion, once at Day 1, Weeks 2, |
| Description: | Day 1, Weeks 2, 6, 14, 22, and 30 along with ciprofloxacin 500 mg, | 6, 14, 22, and 30 along with ciprofloxacin 500 mg, tablet, |
| | tablet, orally twice daily up to Week 4. | orally twice daily up to Week 4. |
| Overall Number of | 50 | 51 |
| Participants Analyzed | | |
| Deviation) | | |
| Unit of Measure: score | | |
| on a scale | | |
| Baseline | 131.5 (30.78) | 137.9 (33.53) |
| Change from Baseline | 14.6 (26.67) | 18.3 (29.20) |
| at Week 14 | | |
| Change from Baseline | 16.0 (29.12) | 21.3 (31.28) |
| at Week 22 | | |
| Change from Baseline | 10.4 (25.98) | 24.4 (34.21) |
| at Week 34 | | |

| ▾ Statistical Analysis 1 | | |
|---|---|---|
| Statistical Analysis | Comparison Group | Placebo IV, Vedolizumab IV 300 mg |
| Overview | Selection | |
| | Comments | Change from Baseline at Week 14 |
| | Type of Statistical Test | Superiority |
| Statistical Test of | P-Value | =0.575 |
| Hypothesis | Comments | No multiplicity adjustment for inferential testing for secondary endpoints was preplanned, p-values from these |
| | | tests are presented as nominal p-values. The significance level was 0.05. |
| | Method | Wilcoxon (Mann-Whitney) |
| | Comments | P-value is from Wilcoxon Rank-sum Test comparing change from Baseline to relevant visits. Wilcoxon-Mann- |
| | | Whitney odds estimator, 95% CI are presented. |
| Method of | Estimation Parameter | Odds Estimator |
| Estimation | Estimated Value | 1.14 |
| | Confidence Interval | (2-Sided) 95% |
| | | 0.61 to 1.67 |
| ▾ Statistical Analysis 2 | | |
| Statistical Analysis | Comparison Group | Placebo IV, Vedolizumab IV 300 mg |
| Overview | Selection | |
| | Comments | Change from Baseline at Week 22 |
| | Type of Statistical Test | Superiority |
| Statistical Test of | P-Value | =0.403 |
| Hypothesis | Comments | No multiplicity adjustment for inferential testing for secondary endpoints was preplanned, p-values from these |
| | | tests are presented as nominal p-values. The significance level was 0.05. |
| | Method | Wilcoxon (Mann-Whitney) |
| | Comments | P-value is from Wilcoxon Rank-sum Test comparing change from Baseline to relevant visits. Wilcoxon-Mann- |
| | | Whitney odds estimator, 95% CI are presented. |
| Method of | Estimation Parameter | Odds Estimator |
| Estimation | Estimated Value | 1.21 |
| | Confidence Interval | (2-Sided) 95% |
| | | 0.65 to 1.78 |
| ▾ Statistical Analysis 3 | | |
| Statistical Analysis | Comparison Group | Placebo IV, Vedolizumab IV 300 mg |
| Overview | Selection | |
| | Comments | Change from Baseline at Week 34 |
| | Type of Statistical Test | Superiority |
| Statistical Test of | P-Value | =0.047 |
| Hypothesis | Comments | No multiplicity adjustment for inferential testing for secondary endpoints was preplanned, p-values from these |
| | | tests are presented as nominal p-values. The significance level was 0.05. |
| | Method | Wilcoxon (Mann-Whitney) |
| | Comments | P-value is from Wilcoxon Rank-sum Test comparing change from Baseline to relevant visits. Wilcoxon-Mann- |
| | | Whitney odds estimator, 95% CI are presented. |
| Method of | Estimation Parameter | Odds Estimator |
| Estimation | Estimated Value | 1.60 |
| | Confidence Interval | (2-Sided) 95% |
| | | 0.85 to 2.34 |

10. Secondary Outcome

| | |
|---|---|
| Title | Change From Baseline in Cleveland Global Quality of Life (CGQL) at Weeks 14, 22, and 34 |
| ▾ Description | The CGQL (Fazio score) is a quality-of-life indicator specifically for participants with ileal pouch-anal anastomosis. |
| | Participants rate 3 items (current quality of life, current quality of health, and current energy level), each on a scale of 0 |
| | to 10 (0 = worst; 10 = best). The scores were added, and the final CGQL utility score was obtained by dividing this result |
| | by 30. The total score ranges from 0 (worst) to 1 (best) where lower scores indicate less quality of life. A negative |
| | change from Baseline indicated worsening. LOCF method was used for analyses. |
| Time Frame | Baseline up to Weeks 14, 22, and 34 |

| ▾ Outcome Measure Data | | |
|---|---|---|
| ▾ Analysis Population Description | | |
| FAS included all randomized participants who received at least 1 dose of study medication, as randomized. Overall number analyzed are the number of | | |
| participants with data evaluable for analyses. | | |
| Arm/Group Title | Placebo IV | Vedolizumab IV 300 mg |
| ▾ Arm/Group | Vedolizumab placebo-matching intravenous (IV) infusion, once at | Vedolizumab 300 mg, IV infusion, once at Day 1, Weeks 2, 6, |
| Description: | Day 1, Weeks 2, 6, 14, 22, and 30 along with ciprofloxacin 500 mg, | 14, 22, and 30 along with ciprofloxacin 500 mg, tablet, orally |
| | tablet, orally twice daily up to Week 4. | twice daily up to Week 4. |
| Arm/Group Title | Placebo IV | Vedolizumab IV 300 mg |
| Overall Number of | 49 | 50 |
| Participants Analyzed | | |
| Mean (Standard | | |
| Deviation) | | |
| Unit of Measure: score | | |
| on a scale | | |
| Baseline | 0.522 (0.1953) | 0.556 (0.1626) |
| Change from Baseline | 0.051 (0.1518) | 0.088 (0.1715) |
| at Week 14 | | |
| Change from Baseline | 0.073 (0.1491) | 0.093 (0.1648) |
| at Week 22 | | |
| Change from Baseline | 0.056 (0.1544) | 0.083 (0.1831) |
| at Week 34 | | |

| ▾ Statistical Analysis 1 | | |
|---|---|---|
| Statistical Analysis | Comparison Group | Placebo IV, Vedolizumab IV 300 mg |
| Overview | Selection | |
| | Comments | Change from Baseline at Week 14 |
| | Type of Statistical Test | Superiority |
| Statistical Test of | P-Value | =0.119 |
| Hypothesis | Comments | No multiplicity adjustment for inferential testing for secondary endpoints was preplanned, p-values from these |
| | | tests are presented as nominal p-values. The significance level was 0.05. |
| | Method | Wilcoxon (Mann-Whitney) |
| | | Whitney odds estimator, 95% CI are presented. |
| Method of | Estimation Parameter | Odds Estimator |
| Estimation | Estimated Value | 1.44 |
| | Confidence Interval | (2-Sided) 95% |
| | | 0.77 to 2.12 |
| ▾ Statistical Analysis 2 | | |
| Statistical Analysis | Comparison Group | Placebo IV, Vedolizumab IV 300 mg |
| | Selection | |
| Overview | Comments | Change from Baseline at Week 22 |
| | Type of Statistical Test | Superiority |
| Statistical Test of | P-Value | =0.542 |
| Hypothesis | Comments | No multiplicity adjustment for inferential testing for secondary endpoints was preplanned, p-values from these |
| | | tests are presented as nominal p-values. The significance level was 0.05. |
| | Method | Wilcoxon (Mann-Whitney) |
| | Comments | P-value is from Wilcoxon Rank-sum Test comparing change from Baseline to relevant visits. Wilcoxon-Mann- |
| | | Whitney odds estimator, 95% CI are presented. |
| Method of | Estimation Parameter | Odds Estimator |
| Estimation | Estimated Value | 1.15 |
| | Confidence Interval | (2-Sided) 95% |
| | | 0.62 to 1.69 |
| ▾ Statistical Analysis 3 | | |
| Statistical Analysis | Comparison Group | Placebo IV, Vedolizumab IV 300 mg |
| Overview | Selection | |
| | Comments | Change from Baseline at Week 34 |
| | Type of Statistical Test | Superiority |
| Statistical Test of | P-Value | =0.404 |
| Hypothesis | Comments | No multiplicity adjustment for inferential testing for secondary endpoints was preplanned, p-values from these |
| | | tests are presented as nominal p-values. The significance level was 0.05. |
| | Method | Wilcoxon (Mann-Whitney) |
| | Comments | P-value is from Wilcoxon Rank-sum Test comparing change from Baseline to relevant visits. Wilcoxon-Mann- |
| | | Whitney odds estimator, 95% CI are presented. |
| Method of | Estimation Parameter | Odds Estimator |
| Estimation | Estimated Value | 1.22 |
| | Confidence Interval | (2-Sided) 95% |
| | | 0.65 to 1.78 |

TABLE 5
Adverse Events

| | |
|---|---|
| Time Frame | From first dose of study drug up to 18 weeks after last dose (Up to 50 weeks) |
| Adverse Event Reporting | At each visit the investigator documented any occurrence of adverse events |
| Description | and abnormal laboratory findings. Any event spontaneously reported by the |
| | participant or observed by the investigator was recorded, irrespective of the |
| | relation to study treatment. |

| Arm/Group Title | Placebo IV | Vedolizumab IV 300 mg |
|---|---|---|
| ▾Arm/Group Description | Vedolizumab placebo-matching IV | Vedolizumab 300 mg, IV infusion, |
| | infusion, once at Day 1, Weeks 2, 6, | once at Day 1, Weeks 2, 6, 14, 22, |
| | 14, 22, and 30 along with | and 30 along with ciprofloxacin 500 |
| | ciprofloxacin 500 mg, tablet, orally | mg, tablet, orally twice daily up to |
| | twice daily up to Week 4. | Week 4. |
| | Placebo IV | Vedolizumab IV 300 mg |
| | Affected/at Risk (%) | Affected/at Risk (%) |

All-Cause Mortality

| | | |
|---|---|---|
| Total | 0/51 (0.00%) | 0/51 (0.00%) |

▾Serious Adverse Events

| | | |
|---|---|---|
| Total | 4/51 (7.84%) | 3/51 (5.88%) |
| Gastrointestinal disorders | | |
| Abdominal pain ^{† 1} | 1/51 (1.96%) | 0/51 (0.00%) |
| Pouchitis ^{† 1} | 1/51 (1.96%) | 2/51 (3.92%) |
| Intestinal obstruction ^{† 1} | 1/51 (1.96%) | 0/51 (0.00%) |
| Infections and infestations | | |
| Gastroenteritis ^{† 1} | 0/51 (0.00%) | 1/51 (1.96%) |
| Neoplasms benign, | | |
| malignant and unspecified | | |
| (incl cysts and polyps) | | |
| Basal cell carcinoma ^{† 1} | 1/51 (1.96%) | 0/51 (0.00%) |

▾Other (Not Including Serious) Adverse Events

| | | |
|---|---|---|
| Frequency Threshold for | 5% | |
| Reporting Other Adverse | | |
| Events | | |
| Total | 35/51 (68.63%) | 42/51 (82.35%) |
| Gastrointestinal disorders | | |
| Pouchitis ^{† 1} | 20/51 (39.22%) | 23/51 (45.10%) |
| Nausea ^{† 1} | 5/51 (9.80%) | 5/51 (9.80%) |
| Abdominal pain ^{† 1} | 2/51 (3.92%) | 4/51 (7.84%) |
| Frequent bowel | 2/51 (3.92%) | 4/51 (7.84%) |
| movements ^{† 1} | | |
| Infections and infestations | | |
| Nasopharyngitis ^{† 1} | 6/51 (11.76%) | 6/51 (11.76%) |
| Influenza ^{† 1} | 1/51 (1.96%) | 4/51 (7.84%) |
| Gastroenteritis ^{† 1} | 3/51 (5.88%) | 1/51 (1.96%) |
| Upper respiratory tract | 1/51 (1.96%) | 5/51 (9.80%) |
| infection ^{† 1} | | |
| Musculoskeletal and | | |
| connective tissue disorders | | |
| Arthralgia ^{† 1} | 9/51 (17.65%) | 7/51 (13.73%) |
| Back pain ^{† 1} | 5/51 (9.80%) | 2/51 (3.92%) |
| Nervous system disorders | | |
| Headache ^{† 1} | 3/51 (5.88%) | 10/51 (19.61%) |
| Respiratory, thoracic and | | |
| mediastinal disorders | | |
| Dyspnoea ^{† 1} | 3/51 (5.88%) | 0/51 (0.00%) |

¹ Term from vocabulary, MedDRA 23.0
^{†} Indicates events were collected by systematic assessment

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The contents of all references, patents and published patent applications cited throughout this application are incorporated herein by reference.

SEQUENCE TABLE

| SEQ ID | | |
|---|---|---|
| NO: | DESCRIPTION | SEQUENCE |
| 1 | Heavy chain (HC) | QVQLVQSGAEVKKPGASV |
| | variable region (amino | KVSCKGSGYTFTSYWMHW |
| | acid) | VRQAPGQRLEWIGEIDPS |
| | | ESNTNYNQKFKGRVTLTV |
| | | DISASTAYMELSSLRSED |
| | | TAVYYCARGGYDGWDYAI |
| | | DYWGQGTLVTVSS |
| 2 | HC CDR1 (amino | SYWMH |
| | acid) | |
| 3 | HC CDR2 (amino | EIDPSESNTNYNQKFKG |
| | acid) | |
| 4 | HC CDR3 (amino | GGYDGWDYAIDY |
| | acid) | |
| 5 | Light chain (LC) | DVVMTQSPLSLPVTPGEP |
| | variable region (amino | ASISCRSSQSLAKSYGNT |
| | acid) | YLSWYLQKPGQSPQLLIY |
| | | GISNRFSGVPDRFSGSGS |
| | | GTDFTLKISRVEAEDVGV |
| | | YYCLQGTHQPYTFGQGTK |
| | | VEIK |
| 6 | LC CDR1 (amino | RSSQSLAKSYGNTYLS |
| | acid) | |
| 7 | LC CDR2 (amino | GISNRFS |
| | acid) | |
| 8 | LC CDR3 (amino | LQGTHQPYT |
| | acid) | |
| 9 | Heavy chain amino | QVQLVQSGAEVKKPGASV |
| | acid sequence | KVSCKGSGYTFTSYWMHW |
| | | VRQAPGQRLEWIGEIDPS |
| | | ESNTNYNQKFKGRVTLTV |
| | | DISASTAYMELSSLRSED |
| | | TAVYYCARGGYDGWDYAI |
| | | DYWGQGTLVTVSSASTKG |
| | | PSVFPLAPSSKSTSGGTA |
| | | ALGCLVKDYFPEPVTVSW |
| | | NSGALTSGVHTFPAVLQS |
| | | SGLYSLSSVVTVPSSSLG |
| | | TQTYICNVNHKPSNTKVD |
| | | KKVEPKSCDKTHTCPPCP |
| | | APELAGAPSVFLFPPKPK |
| | | DTLMISRTPEVTCVVVDV |
| | | SHEDPEVKFNWYVDGVEV |
| | | HNAKTKPREEQYNSTYRV |
| | | VSVLTVLHQDWLNGKEYK |
| | | CKVSNKALPAPIEKTISK |
| | | AKGQPREPQVYTLPPSRD |
| | | ELTKNQVSLTCLVKGFYP |
| | | SDIAVEWESNGQPENNYK |
| | | TTPPVLDSDGSFFLYSKL |
| | | TVDKSRWQQGNVFSCSVM |
| | | HEALHNHYTQKSLSLSPG |
| | | K |
| 10 | Light chain amino | DVVMTQSPLSLPVTPGEP |
| | acid sequence | ASISCRSSQSLAKSYGNT |
| | | YLSWYLQKPGQSPQLLIY |
| | | GISNRFSGVPDRFSGSGS |
| | | GTDFTLKISRVEAEDVGV |
| | | YYCLQGTHQPYTFGQGTK |
| | | VEIKRTVAAPSVFIFPPS |
| | | DEQLKSGTASVVCLLNNF |
| | | YPREAKVQWKVDNALQSG |
| | | NSQESVTEQDSKDSTYSL |
| | | SSTLTLSKADYEKHKVYA |
| | | CEVTHQGLSSPVTKSFNR |
| | | GEC |

1. A method of treating chronic pouchitis in a human subject, said method comprising selecting a human subject having chronic pouchitis and administering a therapeutically effective dose of a humanized anti-α4β7 antibody, or antigen binding fragment thereof, to the subject, such that chronic pouchitis is treated,
wherein the humanized anti-α4β7 antibody, or antigen binding fragment thereof, is an IgG1 or an IgG4 isotype; comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6, and
wherein the human subject had an endoscopic Pouchitis Disease Activity Index (PDAI) subscore of 6 at selection and/or was TNFα naïve at selection.

2. The method of claim 1, wherein the therapeutically effective dose is 108 mg or 300 mg.

3. A method of treating chronic pouchitis in a human subject, said method comprising
selecting a human subject who has chronic pouchitis; and
administering to the human subject an initial dose of 300 mg of a humanized anti-α4β7 antibody, or antigen binding fragment thereof, followed by a subsequent dose of 300 mg of the humanized anti-α4β7 antibody, or antigen binding fragment thereof, at least every two weeks thereafter, such that the chronic pouchitis is treated in the subject,
wherein the humanized anti-α4β7 antibody, or antigen binding fragment thereof, is an IgG1 or an IgG4 isotype; comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

4. A method of treating chronic pouchitis in a human subject, said method comprising
selecting a human subject who has chronic pouchitis;
administering an initial dose of 300 mg of a humanized anti-α4β7 antibody, or an antigen binding fragment thereof, to the human subject:
administering a second dose of 300 mg of the humanized anti-α4β7 antibody, or antigen binding fragment thereof, at about two weeks after the initial dose;
administering a third dose of 300 mg of the humanized anti-α4β7 antibody, or antigen binding fragment thereof, at about six weeks after the initial dose; and
administering a dose of 300 mg of the humanized anti-α4β7 antibody, or antigen binding fragment thereof, every four or eight weeks after the third dose,
wherein the humanized anti-α4β7 antibody, or antigen binding fragment thereof, is an IgG1 or an IgG4 isotype; comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

5. A method of treating chronic pouchitis in a human subject, said method comprising
selecting a human subject who has chronic pouchitis;
administering an initial dose of 300 of a humanized anti-α4β7 antibody, or an antigen binding fragment thereof, to the human subject:
administering a second dose of 300 of the humanized anti-α4β7 antibody, or antigen binding fragment thereof, at about two weeks after the initial dose;
administering a third dose of 300 of the humanized anti-α4β7 antibody, or antigen binding fragment thereof, at about six weeks after the initial dose; and
subcutaneously administering a dose of 108 mg of the humanized anti-α4β7 antibody, or antigen binding fragment thereof, every one or two weeks after the third dose,
wherein the humanized anti-α4β7 antibody, or antigen binding fragment thereof, is an IgG1 or an IgG4 isotype; comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

6. The method of claim 1, wherein the anti-α4β7 antibody, or antigen binding fragment thereof, comprises a heavy chain variable region as set forth in SEQ ID NO: 1 and a light chain variable region as set forth in SEQ ID NO: 5.

7.-9.

10. A method of treating chronic pouchitis in a human subject, said method comprising selecting a subject having chronic pouchitis and administering a therapeutically effective dose of an anti-α4β7 antibody to the subject, such that chronic pouchitis is treated, wherein the anti-α4β7 antibody is vedolizumab, wherein the human subject has an endoscopic PDAI subscore of more than 5 at selection and/or the human subject was TNFα naïve at selection.

11. The method of claim 10, wherein the therapeutically effective dose of vedolizumab is 108 mg or 300 mg.

12. A method of treating chronic pouchitis in a human subject, said method comprising
selecting a human subject who has chronic pouchitis; and
administering to the human subject an initial dose of 300 mg of an anti-4β7-antibody followed a subsequent dose of 300 mg of the anti-α4β7 antibody at least every two weeks thereafter, such that the chronic pouchitis is treated in the subject,
wherein the anti-α4β7 antibody is vedolizumab.

13. The method of claim 12, comprising
administering the initial dose of 300 mg of the anti-α4β7 antibody to the human subject:
administering a second dose of 300 mg of the anti-α4β7 antibody at about two weeks after the initial dose;
administering a third dose of 300 mg of the anti-α4β7 antibody at about six weeks after the initial dose; and
administering one or more subsequent doses of 300 mg of the anti-α4β7-antibody every eight weeks after the third dose,
wherein the anti-α4β7 antibody is vedolizumab.

14. A method of treating chronic pouchitis in a human subject, said method comprising administering to the human subject an anti-α4β7 antibody, or an antigen-binding fragment thereof, at 0 and 2 weeks or 0, 2 and 6 weeks via IV followed 2, 4, 6 or 8 weeks later, by subsequent doses administered every 2 weeks via subcutaneous administration to the human subject having chronic pouchitis.

15. (canceled)

16. The method of claim 14, wherein the IV doses comprise a 300 mg dose of vedolizumab and the subsequent doses comprise a 108 mg dose of vedolizumab.

17. (canceled)

18. The method of claim 1, further comprising administering an antibiotic to the human subject.

19. The method of claim 18, wherein the antibiotic is discontinued by 4 weeks following the initial administration of the anti-α4β7 antibody, or antigen binding fragment thereof,
wherein the antibiotic is ciprofloxacin, or
wherein the antibiotic is administered daily.

20.-21. (canceled)

22. The method of claim 4, wherein the human subject received long-term continuous low-dose antibiotic therapy or received frequent pulse antibiotic, prior to selection;
wherein the human subject had an ileal pouch anal anastomosis (IPAA) at least one year prior to selection.

23. (canceled)

24. The method of claim claim 1, wherein the human subject had moderately to severely active chronic pouchitis, who had undergone proctocolectomy and ileal pouch anal anastomosis for ulcerative colitis.

25. The method of claim 24, wherein the human subject had an inadequate response with or lost response to antibiotic therapy.

26.-27. (canceled)

28. The method of claim 1, wherein the human subject achieves remission of pouchitis.

29. The method of claim 28, wherein the human subject achieves remission by about 14 weeks following the initial dose of the anti-α4β7 antibody or vedolizumab;
wherein remission is defined as pouchitis having a modified Pouchitis Disease Activity Index (mPDAI) of <5 and a reduction in overall mPDAI score of ≥2 from baseline; or
wherein remission is maintained for at least 34 weeks following the initial dose of the anti-α467 antibody, or fragment thereof.

30.-31. (canceled)

32. The method of claim 4, wherein the human subject achieves at least one of the following:
symptomatic remission of pouchitis,
a change in PDAI endoscopic score at weeks 14 and 34 compared to baseline,
a change in PDAI Histologic Findings Score at weeks 14 and 34 compared to baseline, a change in PDAI Score at weeks 14 and 34 compared to baseline,
a change in Inflammatory Bowel Disease Questionnaire (IBDQ) Total Score and Subscale Score at weeks 14, 22 and 34 compared to baseline, or
a change in 3-Item Cleveland Global Quality of Life (CGQL) at weeks 14, 22 and 34 compared to baseline.

33. The method of claim 1, wherein the anti-α4β7 antibody, or fragment thereof, is administered to the human subject intravenously and/or subcutaneously.

34. (canceled)
